(19) United States
(12) Patent Application Publication
(10) Pub. No.: US 2011/0129460 A1
(43) Pub. Date: Jun. 2, 2011

(54) Combination Antibodies For The Treatment And Prevention Of Disease Caused By Bacillus Anthracis And Related Bacteria And Their Toxins

(75) Inventor: Herman Groen, Groningen (NL)

(73) Assignee: IQ Therapeutics B.V., Groningen (NL)

(21) Appl. No.: 12/840,779

(22) Filed: Jul. 21, 2010

Related U.S. Application Data
(63) Continuation-in-part of application No. 12/836,455, filed on Jul. 14, 2010.
Application No. 12/836,455 is a continuation-in-part of application No. PCT/IB2010/000146, filed on Jan. 14, 2010.
(60) Provisional application No. 61/144,507, filed on Jan. 14, 2009.

(51) Int. Cl. A61K 39/40; A61K 39/02; A61P 31/04

(52) U.S. Cl. 424/133.1; 424/150.1; 424/234.1; 424/246.1; 424/142.1

(57) ABSTRACT

The invention relates to methods and compositions for the prevention and treatment of disease caused by B. anthracis or a bacterium which produces toxins or toxin components homologous to the virulence factors produced by B. anthracis or to the toxins or toxin components themselves, in the absence of bacteria. The methods and compositions of the invention comprise a combination of at least two antibodies, preferably monoclonal antibodies, most preferably human monoclonal antibodies, each of which binds with high affinity to a different epitope of one or more bacterial antigens.

### REFERENCE TO RELATED APPLICATIONS

This application is a continuation in part of and claims priority to U.S. Ser. No. 12/836,455, filed Jul. 14, 2010, which is a continuation in part of and claims priority to PCT Application No. PCT/IB2010/000146, filed Jan. 14, 2010, which claims the benefit of U.S. Provisional Application No. 61/144,507, filed Jan. 14, 2009, the contents of each of which are incorporated by reference in their entirety.

### FIELD OF THE INVENTION

The present invention relates to compositions and methods for the treatment and prevention of disease caused by Bacillus anthracis (anthrax) or a bacterium which produces toxins or toxin components homologous to those produced by B. anthracis, or disease caused by the toxins or toxin components themselves, using a combination of at least two neutralizing monoclonal antibodies.

### BACKGROUND OF THE INVENTION

Bacillus anthracis, the etiologic agent of anthrax, is a gram-positive, rod shaped, aerobic and/or facultative anaerobic, spore-forming bacterium that can cause human disease via the gastrointestinal, cutaneous, or inhalation routes. The incubation period usually varies from 12 hours to 5 days depending upon the dose received. The onset can be longer following inhalation exposure and some reports suggest a delayed onset of several weeks in low dose exposure or following removal of therapeutic intervention. With an anthrax inhalation, the initial clinical signs and symptoms are nonspecific and may include malaise, headache, fever, nausea, and vomiting. These are followed by a sudden onset of respiratory distress with dyspnea, stridor, cyanosis and chest pain. The onset of respiratory distress is followed by shock and death with high mortality.

Anthrax is considered a serious biological terrorist and military threat due to the highly lethal effects when exposure is by inhalation (approaching 100 percent lethality) and the stability of the B. anthracis spore. The virulence of B. anthracis is based on two virulence factors: encapsulation (prevention of phagocytosis) and the production of two interlinked toxins, lethal toxin and edema toxin. Three exoprotein components, protective antigen (PA), lethal factor (LF), and edema factor (EF), interact to form the two toxins. PA combines with lethal factor to produce lethal toxin and with edema factor to produce edema toxin. PA binds to host cells and is cleaved, exposing binding sites for which lethal factor and edema factor compete. The current consensus is that the cleaved PA forms a channel into the cell, allowing lethal toxin (PA-LF) or edema toxin (PA-EF) to enter.

The PA monomer consists of four functional domains: domain 1 (residues 1-258), domain 2 (residues 259-487), domain 3 (residues 488-595), and domain 4 (residues 596-735). Domain 1, the amino terminal domain, contains a furin protease cleavage site. Cleavage of Domain 1 releases a 20 kilodalton fragment (PA20) which triggers heptamerization of the remainder of the protein at the cell surface. Domain 2 assists in heptamerization and, along with domain III, forms a heptameric pore on the cell surface that allows binding of LF or EF, enabling endocytosis of the toxin complex into the cell. Domain 4 contains the host cell receptor binding site.

It is generally believed that lethal toxin is responsible for the majority of the tissue damage and systemic shock that occurs as the infection progresses, but the mechanism is not clearly understood. Internalization and translocation of the lethal factor into the cytosol occurs when the PA protein binds to it cell surface receptor. The highly specific LF enzyme has four domains (1-4). Domain III has a hydrophobic core (282-382) and contains a five-tandem repeat 101 amino acid sequence. Assembly and cellular internalization of lethal toxin results in increased permeability to sodium and potassium ions followed by ATP hydrolysis which inhibits macromolecular synthesis and leads to cell death.

As disease progresses, lethal toxin will eventually accumulate to a level at which antibiotics are no longer effective, even though the bacteria is sensitive to the antibiotic. This means that antibiotics treatment must be started during the very early stages of infection in order to potentially be successful. Since a biological attack is likely to occur without warning, such early treatment will often be impossible. It is therefore important to develop methods that neutralize the effects of lethal toxin. One approach is vaccination with toxin components. This approach has the disadvantage of being effective only for those well advanced in a vaccination program (vaccination currently takes approximately 12 months to become effective) and for those with a highly competent immune system. Thus, vaccination is ineffective as a post-exposure means of treatment. Other therapeutic strategies are needed to neutralize the devastating effects of lethal toxin during the post-exposure treatment window.

Bacteria other than B. anthracis may contain B. anthracis virulence genes. In other words, other bacteria may contain genes that produce proteins homologous to those of B. anthracis for encapsulation and the production of toxins, such as PA, LF, and EF. An example is the PA protein of Bacillus cereus G9241 and the homologous proteins of B. thuringiensis and C. perfringens (see Hoffmaster et al., Proc. Natl. Acad. Sci. U.S.A. (2004) 101:8449-8454; Hoffmaster et al., J. Clin. Microbiol. (2006) 44:3352-3360; and Petosa et al., Nature (1997) 385:833-838).

The currently recommended post-exposure treatment for anthrax is a combination of antibiotics (ciprofloxacin or doxycycline), licensed human vaccine (AVA), and, in severe cases, intravenously administered preformed human polyclonal anthrax immunoglobulin (AIGIV) derived from immunized donors. AIGIV has a number of advantages. It provides instant protection, is likely to be effective during mid- to advanced-stage disease, is equally effective against antibiotic-resistant strains, results in minimal adverse reactions, has a prolonged serum half-life, and targets multiple epitopes, making it difficult to subvert its efficacy. However, despite these advantages, AIGIV suffers from several serious drawbacks that prevent its usefulness on a large scale. First, AIGIV therapy requires the maintenance of stocks of antibodies having high toxin neutralization activity. These stocks must be obtained from an immunologically diverse population of donors, and must be constantly renewed.

The present invention provides an alternative approach which utilizes a combination of antibodies with neutralizing activity against both protective antigen and lethal factor for the prevention and treatment of disease caused by Bacillus anthracis or a bacterium which produces toxins or toxin components homologous to those produced by B. anthracis, or disease caused by the toxins or toxin components themselves.

### SUMMARY OF THE INVENTION

The present invention provides methods and compositions for the treatment and prevention of disease caused by bacterial infection, particularly infection by B. anthracis or a bacterium which produces toxins or toxin components homologous to those produced by B. anthracis, or disease caused by the toxins or toxin components themselves. The methods and compositions of the invention comprise a combination of at least two neutralizing antibodies, preferably monoclonal antibodies, most preferably human monoclonal antibodies, each of which binds to a different bacterial antigen. Preferably, the antigens are selected from the protective antigen (PA), lethal factor (LF), and edema factor (EF) of B. anthracis, or a homolog of any of the foregoing.

The methods and compositions of the invention offer enhanced protection against infection when administered prophylactically and provide an increased probability of survival when administered therapeutically. The approach of combining at least two antibodies having different antigen specificities provides broader protection than a single antibody or single antigen approach. The methods and compositions of the invention are more likely than single antibody approaches to be effective against B. anthracis, including variations in bacterial strains and escape mutants, as well as against other bacteria which produce toxins or toxin components homologous to those produced by B. anthracis. In addition, the methods and compositions of the invention advantageously extend the treatment window for subjects exposed to B. anthracis, or to bacteria which produce toxins or toxin components homologous to those produced by B. anthracis, or to the toxins or toxin components themselves, in the absence of bacteria, thereby improving the probability of survival. The compositions and methods of the invention also provide significant cost reductions and reduced health risks compared to mass vaccination strategies because the present invention targets treatment to those who have been exposed or are likely to be exposed to B. anthracis toxins, toxin components, or homologs thereof.

The invention provides a method for the treatment of disease caused by B. anthracis toxins, toxin components, or homologs thereof, in a subject in need of such treatment comprising administering to the subject at least two neutralizing monoclonal antibodies, or antigen binding fragments thereof, wherein each of the antibodies has affinity for a different bacterial antigen selected from the protective antigen (PA), lethal factor (LF), and edema factor (EF) of B. anthracis, or a homolog of any of the foregoing. In a preferred embodiment, one of the at least two antibodies has affinity for an epitope of PA within domain 4 of PA, most preferably within amino acid residues 679-693 of domain 4. In another preferred embodiment, one of the at least two antibodies has affinity for an epitope of LF within domain 1 of LF.

In one embodiment, the disease is caused by a bacterium. As used herein, a bacterium or bacteria also include, but are not limited to, bacterial spores of the bacterium or bacteria. In a specific embodiment, the disease is caused by a bacterium selected from the group consisting of B. anthracis, B. cereus, B. thuringiensis, and C. perfringens. In one embodiment, the disease is caused by a bacterium, or a combination of different bacteria which produce one or more proteins homologous to one or more of the PA, LF, and EF proteins of B. anthracis. In another embodiment, the disease results from toxemia caused by one or more bacterial toxins comprising one or more of PA, LF and EF, or a homolog of any of the foregoing. In accordance with this embodiment, toxemia may occur in the presence or absence of bacteria.

In one embodiment, the antibodies are human monoclonal antibodies. In another embodiment, the antibodies are humanized monoclonal antibodies.

In one embodiment, the affinity (Kₐ) of the antibody for its antigen is from 10⁷ M⁻¹ to 10¹⁰ M⁻¹. Preferably, the affinity (Kₐ) of the antibody for its antigen is from 10⁹ M⁻¹ to 10¹⁰ M⁻¹. In one embodiment, the affinity (Kₐ) of each antibody for its antigen is from 10⁷ M⁻¹ to 10¹⁰ M⁻¹. Preferably, the affinity (Kₐ) of each antibody for its antigen is from 10⁹ M⁻¹ to 10¹⁰ M⁻¹.

In one embodiment, one of the at least two antibodies is an anti-PA antibody which neutralizes the protective antigen protein of B. anthracis, or a homolog thereof. In one embodiment, the anti-PA antibody competitively inhibits the binding of the protective antigen protein of B. anthracis, or a homolog thereof, to the monoclonal antibody IQNPA. In another embodiment, the anti-PA antibody competitively inhibits the binding of a polypeptide comprising SEQ ID NO:17 or 18 to the monoclonal antibody IQNPA.

In one embodiment, the anti-PA antibody comprises a variable heavy chain domain (VH) having three complementarity determining regions (CDR), each CDR comprising the following amino acid sequence: VH CDR1: KKPGA (SEQ ID NO:5); VH CDR2: SNAIQWVRQAPGQRLEW (SEQ ID NO:6); and VH CDR3: YMELSSLR (SEQ ID NO:7). In another embodiment, the anti-PA antibody comprises a variable light chain domain (VL) having three CDRs, each CDR comprising the following amino acid sequence: VL CDR1: LTQSPGTLSLS (SEQ ID NO:8); VL CDR2: SYSSLAW (SEQ ID NO:9); and VL CDR3: GPDFTLTIS (SEQ ID NO:10). In a particular embodiment, the anti-PA antibody comprises six CDRs, each comprising the following amino acid sequence: VH CDR1: SEQ ID NO:5, VH CDR2: SEQ ID NO:6, VH CDR3: SEQ ID NO:7, VL CDR1: SEQ ID NO:8, VL CDR2: SEQ ID NO:9, and VL CDR3: SEQ ID NO:10.

In a specific embodiment, the anti-PA antibody is the human monoclonal antibody IQNPA.

In one embodiment, one of the at least two antibodies is an anti-LF antibody which neutralizes the lethal factor protein of B. anthracis, or a homolog thereof. In one embodiment, the anti-LF antibody competitively inhibits the binding of the lethal factor protein of B. anthracis, or a homolog thereof, to the monoclonal antibody IQNLF. In another embodiment, the anti-LF antibody competitively inhibits the binding of a polypeptide comprising SEQ ID NO:19 to the monoclonal antibody IQNLF.

In one embodiment, the anti-LF antibody comprises a variable heavy chain domain (VH) having three complementarity determining regions (CDR), each CDR comprising the following amino acid sequence: VH CDR1: VQPGG (SEQ ID NO:11), VH CDR2: SYAMSWVRQAPGKGLEW (SEQ ID NO:12), and VH CDR3: YMQMNSL (SEQ ID NO:13). In another embodiment, the anti-LF antibody comprises a variable light chain domain (VL) having three CDRs, each CDR comprising the following amino acid sequence: VL CDR1: TQSPDFQSVSP (SEQ ID NO:14), VL CDR2: SSLHWYQ (SEQ ID NO:15), and VL CDR3: DFTLTINSL (SEQ ID NO:16). In a particular embodiment, the antibody comprises six CDRs, each comprising the following amino acid sequence: VH CDR1: SEQ ID NO:11, VH CDR2: SEQ ID NO:12, VH CDR3: SEQ ID NO:13, VL CDR1: SEQ ID NO:14, VL CDR2: SEQ ID NO:15, and VL CDR3: SEQ ID NO:16.

In a specific embodiment, the anti-LF antibody is the human monoclonal antibody IQNLF.

In one embodiment, one of the at least two neutralizing monoclonal antibodies is an anti-PA antibody which neutralizes the protective antigen protein of B. anthracis, or a homolog thereof, and the other antibody is an anti-LF antibody which neutralizes the lethal factor protein of B. anthracis, or a homolog thereof. In a specific embodiment, the antibodies are the human monoclonal antibodies, IQNPA and IQNLF.

In one embodiment, each antibody is administered at a dose of from 1 to 20 mg/kg body weight of the subject. In another embodiment, one antibody is administered at a dose of from 1 to 10 mg/kg body weight of the subject. In another embodiment, one antibody is administered at a dose of from 2.5 to 15 mg/kg body weight of the subject. In one embodiment, the doses of the at least two antibodies are administered separately. In another embodiment, the doses of the at least two antibodies are administered at substantially the same time. In certain embodiments, each dose is in a separate composition. In other embodiments, the doses are contained in the same composition.

In one embodiment, the antibodies are administered to the subject before the subject's exposure to a bacterium (e.g., B. anthracis, B. cereus, B. thuringiensis, and C. perfringens), or B. anthracis toxins, toxin components, or homologs thereof. In one embodiment, the antibodies are administered to the subject at least 1 hour, at least 2 hours, at least 4 hours, at least 8 hours, at least 12 hours, at least 24 hours, at least 36 hours, at least 48 hours, at least 60 hours, at least 72 hours or at least 96 hours before the subject's exposure to a bacterium (e.g., B. anthracis, B. cereus, B. thuringiensis, and C. perfringens), or B. anthracis toxins, toxin components, or homologs thereof. In one embodiment, the antibodies are administered to the subject at least 5 days, at least 6 days, at least 7 days, at least 8 days, at least 9 days, at least 10 days, at least 12 days or at least 14 days before the subject's exposure to a bacterium (e.g., B. anthracis, B. cereus, B. thuringiensis, and C. perfringens), or B. anthracis toxins, toxin components, or homologs thereof.

In one embodiment, the antibodies are administered to the subject after the subject's exposure to a bacterium (e.g., B. anthracis, B. cereus, B. thuringiensis, and C. perfringens), or B. anthracis toxins, toxin components, or homologs thereof. In one embodiment, the antibodies are administered to the subject at least 1 hour, at least 2 hours, at least 4 hours, at least 8 hours, at least 12 hours, at least 24 hours, at least 36 hours, at least 48 hours, at least 60 hours, at least 72 hours or at least 96 hours after the subject's exposure to a bacterium (e.g., B. anthracis, B. cereus, B. thuringiensis, and C. perfringens), or B. anthracis toxins, toxin components, or homologs thereof. In one embodiment, the antibodies are administered to the subject at least 5 days, at least 6 days, at least 7 days, at least 8 days, at least 9 days, at least 10 days, at least 12 days or at least 14 days after the subject's exposure to a bacterium (e.g., B. anthracis, B. cereus, B. thuringiensis, and C. perfringens), or B. anthracis toxins, toxin components, or homologs thereof. In one embodiment, the antibodies are administered to the subject between 0 and 48 hours after the subject's exposure to a bacterium (e.g., B. anthracis, B. cereus, B. thuringiensis, and C. perfringens), or B. anthracis toxins, toxin components, or homologs thereof. In another embodiment, the antibodies are administered to the subject at least 48 hours after the subject's exposure to a bacterium (e.g., B. anthracis, B. cereus, B. thuringiensis, and C. perfringens), or B. anthracis toxins, toxin components, or homologs thereof.

In one embodiment, the antibodies are administered to the subject before the subject develops any symptom after the subject is exposed to a bacterium (e.g., B. anthracis, B. cereus, B. thuringiensis, and C. perfringens), or B. anthracis toxins, toxin components, or homologs thereof. In one embodiment, the antibodies are administered to the subject at least 1 hour, at least 2 hours, at least 4 hours, at least 8 hours, at least 12 hours, at least 24 hours, at least 36 hours, at least 48 hours, at least 60 hours, at least 72 hours or at least 96 hours before the subject develops any symptom after the subject is exposed to a bacterium (e.g., B. anthracis, B. cereus, B. thuringiensis, and C. perfringens), or B. anthracis toxins, toxin components, or homologs thereof. In one embodiment, the antibodies are administered to the subject between 0 and 48 hours before the subject develops any symptom after the subject is exposed to a bacterium (e.g., B. anthracis, B. cereus, B. thuringiensis, and C. perfringens), or B. anthracis toxins, toxin components, or homologs thereof. In another embodiment, the antibodies are administered to the subject 48 hours before the subject develops any symptom after the subject is exposed to a bacterium (e.g., B. anthracis, B. cereus, B. thuringiensis, and C. perfringens), or B. anthracis toxins, toxin components, or homologs thereof.

In one embodiment, the antibodies are administered to the subject after the subject develops a symptom to a bacterium (e.g., B. anthracis, B. cereus, B. thuringiensis, and C. perfringens), or B. anthracis toxins, toxin components, or homologs thereof. In one embodiment, the antibodies are administered to the subject at least 1 hour, at least 2 hours, at least 4 hours, at least 8 hours, at least 12 hours, at least 24 hours, at least 36 hours, at least 48 hours, at least 72 hours or at least 96 hours, after the subject develops a symptom to a bacterium (e.g., B. anthracis, B. cereus, B. thuringiensis, and C. perfringens), or B. anthracis toxins, toxin components, or homologs thereof. In one embodiment, the antibodies are administered to the subject between 0 and 48 hours after the subject develops a symptom to a bacterium (e.g., B. anthracis, B. cereus, B. thuringiensis, and C. perfringens), or B. anthracis toxins, toxin components, or homologs thereof. In another embodiment, the antibodies are administered to the subject 48 hours after the subject develops a symptom to a bacterium (e.g., B. anthracis, B. cereus, B. thuringiensis, and C. perfringens), or B. anthracis toxins, toxin components, or homologs thereof.

Exposure may be in the form of exposure to B. anthracis or to a bacterium that produces toxins or toxin components homologous to those produced by B. anthracis. In an alternative embodiment, such exposure is in the form of exposure to the B. anthracis toxins or toxin components themselves, or homologs thereof, in the absence of bacteria.

In one embodiment, the method further comprises administering to the subject an antibacterial agent. In a specific embodiment, the antibacterial agent is levofloxacin, ciprofloxacin, or doxycycline.

The invention also provides a method for the prevention of disease caused by B. anthracis toxins, toxin components, or homologs thereof, in a subject in need of such prevention comprising administering to the subject at least two neutralizing monoclonal antibodies, or antigen binding fragments thereof, wherein each of the antibodies has affinity for a different bacterial antigen selected from the protective antigen (PA), lethal factor (LF), and edema factor (EF) of B. anthracis, or a homolog of any of the foregoing, and wherein the antibodies are administered prior to the subject's exposure to the B. anthracis toxins, toxin components, or homologs thereof.

In one embodiment, the disease caused by a bacterium. In a specific embodiment, the disease is caused by a bacterium selected from the group consisting of B. anthracis, B. cereus, B. thuringiensis, and C. perfringens. In other embodiments, the disease is caused by a bacterium, or a combination of different bacteria, which produce factors homologous to one or more of the PA, LF, and EF proteins of B. anthracis. In another embodiment, the disease results from toxemia caused by one or more bacterial toxins comprising one or more of PA, LF and EF, or a homolog of any of the foregoing. In accordance with this embodiment, toxemia may occur in the presence or absence of bacteria.

In one embodiment, the antibodies are human monoclonal antibodies. In another embodiment, the antibodies are humanized monoclonal antibodies.

In one embodiment, the affinity (Kₐ) of each antibody for its antigen is from 10⁷ M⁻¹ to 10¹⁰ M⁻¹. Preferably, the affinity (Kₐ) of each antibody for its antigen is from 10⁹ M⁻¹ to 10¹⁰ M⁻¹.

In one embodiment, one of the at least two antibodies is an anti-PA antibody which neutralizes the protective antigen protein of B. anthracis, or a homolog thereof. In another embodiment, the anti-PA antibody competitively inhibits the binding of the protective antigen protein of B. anthracis, or a homolog thereof, to the monoclonal antibody IQNPA. In another embodiment, the anti-PA antibody competitively inhibits the binding of a polypeptide comprising SEQ ID NO:17 or 18 to the monoclonal antibody IQNPA.

In one embodiment, the anti-PA antibody comprises a variable heavy chain domain (VH) having three complementarity determining regions (CDR), each CDR comprising the following amino acid sequence: VH CDR1: KKPGA (SEQ ID NO:5); VH CDR2: SNAIQWVRQAPGQRLEW (SEQ ID NO:6); and VH CDR3: YMELSSLR (SEQ ID NO:7). In another embodiment, the anti-PA antibody comprises a variable light chain domain (VL) having three CDRs, each CDR comprising the following amino acid sequence: VL CDR1: LTQSPGTLSLS (SEQ ID NO:8); VL CDR2: SYSSLAW (SEQ ID NO:9); and VL CDR3: GPDFTLTIS (SEQ ID NO:10). In a particular embodiment, the anti-PA antibody comprises six CDRs, each comprising the following amino acid sequence: VH CDR1: SEQ ID NO:5, VH CDR2: SEQ ID NO:6, VH CDR3: SEQ ID NO:7, VL CDR1: SEQ ID NO:8, VL CDR2: SEQ ID NO:9, and VL CDR3: SEQ ID NO:10.

In a specific embodiment, the anti-PA antibody is the human monoclonal antibody IQNPA.

In one embodiment, one of the at least two antibodies is an anti-LF antibody which neutralizes the lethal factor protein of B. anthracis, or a homolog thereof. In another embodiment, the anti-LF antibody competitively inhibits the binding of the lethal factor protein of B. anthracis, or a homolog thereof, to the monoclonal antibody IQNLF. In another embodiment, the anti-LF antibody competitively inhibits the binding of a polypeptide comprising SEQ ID NO:19 to the monoclonal antibody IQNLF.

In one embodiment, the anti-LF antibody comprises a variable heavy chain domain (VH) having three complementarity determining regions (CDR), each CDR comprising the following amino acid sequence: VH CDR1: VQPGG (SEQ ID NO:11), VH CDR2: SYAMSWVRQAPGKGLEW (SEQ ID NO:12), and VH CDR3: YMQMNSL (SEQ ID NO:13). In another embodiment, the anti-LF antibody comprises a variable light chain domain (VL) having three CDRs, each CDR comprising the following amino acid sequence: VL CDR1: TQSPDFQSVSP (SEQ ID NO:14), VL CDR2: SSLHWYQ (SEQ ID NO:15), and VL CDR3: DFTLTINSL (SEQ ID NO:16). In a particular embodiment, the antibody comprises six CDRs, each comprising the following amino acid sequence: VH CDR1: SEQ ID NO:11, VH CDR2: SEQ ID NO:12, VH CDR3: SEQ ID NO:13, VL CDR1: SEQ ID NO:14, VL CDR2: SEQ ID NO:15, and VL CDR3: SEQ ID NO:16.

In a specific embodiment, the anti-LF antibody is the human monoclonal antibody IQNLF.

In one embodiment, one of the at least two monoclonal antibodies is an anti-PA antibody which neutralizes the protective antigen protein of B. anthracis, or a homolog thereof, and the other antibody is an anti-LF antibody which neutralizes the lethal factor protein of B. anthracis, or a homolog thereof. In a specific embodiment, the antibodies are the human monoclonal antibodies, IQNPA and IQNLF.

In one embodiment, each antibody is administered at a dose of from 1 to 20 mg/kg body weight of the subject. In another embodiment, one antibody is administered at a dose of from 1 to 10 mg/kg body weight of the subject. In another embodiment, one antibody is administered at a dose of from 2.5 to 15 mg/kg body weight of the subject. In one embodiment, the doses of the at least two antibodies are administered separately. In another embodiment, the doses of the at least two antibodies are administered at substantially the same time. In certain embodiments, each dose is in a separate composition. In other embodiments, the doses are contained in the same composition.

The invention also provides a pharmaceutical composition comprising at least two neutralizing monoclonal antibodies, or antigen binding fragments thereof, wherein each of the antibodies has affinity for a different bacterial antigen selected from the protective antigen (PA), lethal factor (LF), and edema factor (EF) of B. anthracis, or a homolog of any of the foregoing, and a pharmaceutically acceptable excipient or carrier.

In one embodiment, the composition comprises an anti-PA antibody which neutralizes the protective antigen protein of B. anthracis, or a homolog thereof. In one embodiment, the anti-PA antibody competitively inhibits the binding of the protective antigen protein of B. anthracis, or a homolog thereof, to the monoclonal antibody IQNPA. In another embodiment, the anti-PA antibody competitively inhibits the binding of a polypeptide comprising SEQ ID NO:17 or 18 to the monoclonal antibody IQNPA. In one embodiment, the anti-PA antibody comprises a variable heavy chain domain (VH) having three complementarity determining regions (CDR), each CDR comprising the following amino acid sequence: VH CDR1: KKPGA (SEQ ID NO:5); VH CDR2: SNAIQWVRQAPGQRLEW (SEQ ID NO:6); and VH CDR3: YMELSSLR (SEQ ID NO:7). In another embodiment, the anti-PA antibody comprises a variable light chain domain (VL) having three CDRs, each CDR comprising the following amino acid sequence: VL CDR1: LTQSPGTLSLS (SEQ ID NO:8); VL CDR2: SYSSLAW (SEQ ID NO:9); and VL CDR3: GPDFTLTIS (SEQ ID NO:10). In a particular embodiment, the anti-PA antibody comprises six CDRs, each comprising the following amino acid sequence: VH CDR1: SEQ ID NO:5, VH CDR2: SEQ ID NO:6, VH CDR3: SEQ ID NO:7, VL CDR1: SEQ ID NO:8, VL CDR2: SEQ ID NO:9, and VL CDR3: SEQ ID NO:10. In a specific embodiment, the anti-PA antibody is the human monoclonal antibody IQNPA.

In another embodiment, the composition comprises an anti-LF antibody which neutralizes the lethal factor protein of B. anthracis, or a homolog thereof. In one embodiment, the anti-LF antibody competitively inhibits the binding of the lethal factor protein of B. anthracis, or a homolog thereof, to the monoclonal antibody IQNLF. In another embodiment, the anti-LF antibody competitively inhibits the binding of a polypeptide comprising SEQ ID NO:19 to the monoclonal antibody IQNLF. In one embodiment, the anti-LF antibody comprises a variable heavy chain domain (VH) having three complementarity determining regions (CDR), each CDR comprising the following amino acid sequence: VH CDR1: VQPGG (SEQ ID NO:11), VH CDR2: SYAMSWVRQAPGKGLEW (SEQ ID NO:12), and VH CDR3: YMQMNSL (SEQ ID NO:13). In another embodiment, the anti-LF antibody comprises a variable light chain domain (VL) having three CDRs, each CDR comprising the following amino acid sequence: VL CDR1: TQSPDFQSVSP (SEQ ID NO:14), VL CDR2: SSLHWYQ (SEQ ID NO:15), and VL CDR3: DFTLTINSL (SEQ ID NO:16). In a particular embodiment, the antibody comprises six CDRs, each comprising the following amino acid sequence: VH CDR1: SEQ ID NO:11, VH CDR2: SEQ ID NO:12, VH CDR3: SEQ ID NO:13, VL CDR1: SEQ ID NO:14, VL CDR2: SEQ ID NO:15, and VL CDR3: SEQ ID NO:16.

In a specific embodiment, the composition comprises the monoclonal IQNPA antibody and the monoclonal IQNLF antibody.

In one embodiment, one of the at least two neutralizing monoclonal antibodies in the composition is an anti-PA antibody which neutralizes the protective antigen protein of B. anthracis, or a homolog thereof, and the other antibody is an anti-LF antibody which neutralizes the lethal factor protein of B. anthracis, or a homolog thereof. In a specific embodiment, the antibodies are the human monoclonal antibodies, IQNPA and IQNLF.

In one embodiment, the composition further comprises at least one antibacterial agent. Preferably, the at least one antibacterial agent is selected from ciprofloxacin, doxycycline, or levofloxacin.

### BRIEF DESCRIPTION OF THE FIGURES

FIG. 1: Kaplan-Meier curves representing time-to-death and survival data for each group of animals in Example 1.4 (Pre-Exposure Efficacy).

FIG. 2: Pharmacokinetic profiles of IQNPA and IQNLF antibodies as determined in diluted rabbit serum. Concentrations were back-calculated from the ELISA values using a 4 parameter fit method and then expressed as ng/mL in 100% rabbit serum.

FIG. 3: Kaplan-Meier curves representing time-to-death and survival data for each group of animals in Example 1.5 (Post-Exposure Efficacy—Experiment 1). IQNPA: red, Groups 1-3; IQNLF: blue, Groups 4-6; IQNPA+IQNLF: green, Groups 8-12; control: gray, Group 7.

FIG. 4: Kaplan-Meier curves representing time-to-death and survival data for each group of animals in Example 1.6 (Post-Exposure Efficacy—Experiment 2).

FIG. 5: Estimated logistic regression curves for each treatment (IQNLF and IQNPA+IQNLF) in Example 1.6. Points show the proportion of animals that survived for each group.

FIG. 6: Estimated logistic regression curves for the IQNLF and combined treatments in Example 1.6. Points show the proportion of animals that survived for each group.

FIG. 7: Estimated logistic regression curves for each treatment (IQNPA, IQNLF, IQNPA+IQNLF) in Example 1.7 (Post-Exposure Efficacy—Experiment 3).

FIG. 8: Kaplan-Meier curves representing time-to-death and survival data for each group in Example 1.7.

FIG. 9: Estimated logistic regression curves for each treatment (IQNLF or IQNPA+IQNLF) in Example 1.8. Points show the proportion of animals that survived for each dose group and treatment involving IQNLF.

FIG. 10: Estimated logistic regression curves for each treatment (IQNPA or IQNPA+IQNLF) in Example 1.8. Points show the proportion of animals that survived for each dose group and treatment involving IQNPA.

FIG. 11: Kaplan-Meier curves representing time-to-death and survival data for each group of animals in Example 1.10 (Post-Exposure Efficacy—Experiment 4).

### DETAILED DESCRIPTION OF THE INVENTION

The methods and compositions of the invention offer enhanced protection against bacterial infection or toxemia (which may occur in the presence or absence of a bacterial infection) caused by B. anthracis or a bacterium which produces toxins or toxin components homologous to those produced by B. anthracis, or disease caused by the toxins or toxin components themselves, when administered before exposure and provide an increased probability of survival when administered following exposure to the bacteria, bacterial toxins, or their component proteins. The invention combines at least two neutralizing monoclonal antibodies, each having a different antigen specificity. Preferably, each of the at least two antibodies has affinity for a different bacterial antigen selected from the protective antigen (PA), lethal factor (LF), and edema factor (EF) of B. anthracis, or a homolog of any of the foregoing.

In one embodiment, at least one of the antibodies binds to an epitope of the PA protein of B. anthracis, or a homolog thereof, that includes one or more amino acids within one of the following groups of amino acids (with reference to Genebank Accession No. PI3423): Group 1 (amino acids 121-150); Group 2 (amino acids 143-158); Group 3 (amino acids 421-440); Group 4 (amino acids 339-359) and Group 5 (amino acids 678-697). In a preferred embodiment, at least one of the antibodies binds to an epitope of PA that includes one or more amino acids within at least one of the following groups of amino acids (with reference to Genebank Accession No. PI3423): Group 6 (Phe-342, Phe-343, Asp-344); Group 7 (Trp-375, Met-379, and Leu-381); Group 8 (Phe-581, Phe-583, Ile-591, Leu-595, and Ile-603); Group 9 (Pro-213, Leu-216, Phe-231, Leu-232, Pro-234, Ile-236, Ile-239, Trp-255, and Phe-265) and Group 10 (Asn-686 and any residue from Lys-708 to Asn-722). In another embodiment, at least one of the antibodies binds to an epitope of LF that includes one or more amino acids within any of domains 1 to 4 of the LF protein of B. anthracis, or a homolog thereof.

As used herein, an “epitope” or an “antigen epitope” may comprise or consist of one or more linear polypeptide fragments of a protein. Different epitopes may comprise or consist of different linear polypeptide fragments of the same protein or different proteins.

As used herein, “neutralizes” or “neutralizing” in the context of antibodies against a bacterium, or against a bacterial toxin or its component, means that the antibody inhibits the ability of the bacterium or the toxin to cause disease. The neutralizing activity of an antibody derives from its ability to bind to a bacterial antigen, particularly a bacterial protein necessary for virulence. In the context of toxins and their components, the antibodies may neutralize, for example, by preventing or reversing the assembly of toxin components to form a functional toxin, or by disabling the toxin or toxin component from exerting its biological activity. For example, in the case of the PA toxin, an antibody may inhibit cleavage of the PA monomer, or it may inhibit the formation of the PA heptamer, or the antibody may block the binding of LF or EF to the PA heptamer. The neutralizing activity of an antibody can be measured, for example, as the ability of the antibody to block entry of the bacteria into cells, to block replication of the bacteria within cells, to enhance the uptake and/or intracellular killing of the bacteria by cells of the immune system, such as macrophages, as well as the ability of the antibody to prevent or ameliorate the clinical symptoms of disease caused by bacterial infection and/or toxemia in a mammal. The neutralizing activity of an antibody against a bacterial toxin can also be measured more directly, for example, using a toxin neutralization assay. Such assays are known in the art and are described, for example in Albrecht et al., Infect. Immunity, (2007) 75:5425-5433 and Li et al., J. Immunol. Methods, (2008) 333:89-106.

As used herein, the term “homolog” refers to a protein having an amino acid sequence which differs from the sequence of the corresponding B. anthracis protein, PA, LF, or EF, but in which the differences are such that the protein retains the function and/or antigenic character of the corresponding B. anthracis protein. Thus, a homolog of PA, LF, or EF may be produced by a bacteria other than B. anthracis. Homology is typically determined on the basis of sequence similarity or sequence identity. In certain embodiments, a homologous protein is one which shares at least 70%, at least 80%, at least 90%, or at least 95% sequence identity over its entire length to a B. anthracis protein selected from PA, LF, and EF. Most preferably, the homolog is at least 98% identical over its entire length to the corresponding B. anthracis protein. In other embodiments, the homologous protein shares high sequence identity to a B. anthracis protein selected from PA, LF, and EF, over one or more regions smaller than its entire length. Preferably, these regions correspond to one or more functional domains. Thus, in one embodiment, a PA homolog shares at least 70%, at least 80%, at least 90%, at least 95%, or at least 98% sequence identity to the PA protein of B. anthracis in one or more functional domains selected from the group consisting of domain 1 (residues 1-258), domain 2 (residues 259-487), domain 3 (residues 488-595), and domain 4 (residues 596-735), with reference to the amino acid sequence of the PA protein of B. anthracis given in GENBANK ACCESSION NO: PI3423. In another embodiment, an LF homolog shares at least 70%, at least 80%, at least 90%, at least 95%, or at least 98% sequence identity to the LF protein of B. anthracis in one or more functional domains selected from the group consisting of domain 1, 2, 3, and 4, with reference to the amino acid sequence of the LF protein of B. anthracis given in GENBANK ACCESSION NO: YP_{—}016503.

Also encompassed are derivatives and analogs of the B. anthracis proteins PA, LF, and EF, and their homologs. Such derivatives and analogs may be full length or other than full length, if the derivative or analog contains a modified amino acid. Derivatives or analogs include, e.g., molecules including regions that are substantially homologous to the PA, LF, or EF proteins, in various embodiments, by at least about 70%, 80%, or 95%, 98%, or even 99% identity over an amino acid sequence of identical size or when compared to an aligned sequence in which the alignment is done using sequence analysis software, such as, for example, the Sequence Analysis Software Package of the Genetics Computer Group, University of Wisconsin Biotechnology Center, 1710 University Avenue, Madison, Wis. 53705, with the default parameters therein.

In the case of polypeptide sequences which are less than 100% identical to a reference B. anthracis sequence, the non-identical positions are preferably, but not necessarily, conservative substitutions of the corresponding residue(s) in the reference sequence. Conservative substitutions typically include substitutions within the following groups: glycine and alanine; valine, isoleucine, and leucine; aspartic acid and glutamic acid; asparagine and glutamine; serine and threonine; lysine and arginine; and phenylalanine and tyrosine. Conservative amino acid changes also refer to changes between amino acids of broadly similar molecular properties, e.g, substitutions within the aliphatic group alanine, valine, leucine and isoleucine. A substitution of glycine for an aliphatic amino acid is also a conservative substitution. Other conservative substitutions include those within the sulfur-containing group methionine and cysteine. Preferred conservative substitution groups are aspartate-glutamate; asparagine-glutamine; valine-leucine-isoleucine; alanine-valine; phenylalanine-tyrosine; and lysine-arginine. Preferably, a substitution other than a conservative amino acid substitution is made outside of a functional domain of the reference protein, e.g., outside of domains 1-4 of either PA or LF.

Where a particular polypeptide is said to have a specific percent identity to a reference polypeptide of a defined length, the percent identity is relative to the reference peptide. Thus, a peptide that is 50% identical to a reference polypeptide that is 100 amino acids long can be a 50 amino acid polypeptide that is completely identical to a 50 amino acid long portion of the reference polypeptide. It might also be a 100 amino acid long polypeptide, which is 50% identical to the reference polypeptide over its entire length. Of course, other polypeptides will meet the same criteria.

The skilled artisan will appreciate that bacterial strains other than B. anthracis may contain B. anthracis virulence genes. In other words, other bacterial strains may contain genes that produce virulence proteins which are the same or homologous to those proteins of B. anthracis which are responsible for virulence. For example, other bacterial strains may produce proteins identical or homologous to the PA, LF, or EF proteins produced by B. anthracis. Accordingly, the antibodies for use in the methods and compositions of the invention include antibodies that neutralize bacteria other than B. anthracis. The dual antibody approach of the present invention can thus be used in the prophylaxis and treatment of disease caused by such other bacteria, including, but not limited to, B. thuringiensis, C. perfringens, and B. cereus, as well as for the prophylaxis and treatment of disease resulting from toxemia caused by exposure to bacterial toxins or toxin components that are identical or homologous to the PA, LF, and/or EF proteins of B. anthracis.

Preferably, the antibodies for use in the methods and compositions of the invention bind to at least one, and most preferably two, of the B. anthracis toxin components, PA, LF, and EF, or a homolog of any of the foregoing. Thus, in a preferred embodiment, the methods and compositions of the invention provide a combination of at least two antibodies, each antibody having affinity for a different antigen selected from the B. anthracis toxin components, PA, LF, and EF, or a homolog of any of the foregoing. Preferably, at least one antibody has affinity for PA, or a homolog thereof, and another antibody has affinity for LF, or a homolog thereof.

### 1.1 Antibodies

The antibodies for use in the methods and compositions of the invention are monoclonal antibodies. The terms “antibody” and “antibodies” refer to fully human antibodies, humanized antibodies, camelised antibodies, chimeric antibodies, CDR-grafted antibodies, single-chain Fvs (scFv), disulfide-linked Fvs (sdFv), Fab fragments, F(ab′) fragments, and antigen-binding fragments of any of the foregoing. In particular, the antibodies include immunoglobulin molecules and antigen-binding active fragments of immunoglobulin molecules, i.e., molecules that contain an antigen binding site. Such fragments may or may not be fused to another immunoglobulin domain including, but not limited to, an Fc region or fragment thereof. The skilled person will appreciate that other fusion products may be generated, including but not limited to, scFv-Fc fusions, variable region (e.g., VL and VH)-Fc fusions, and scFv-scFv-Fc fusions. Immunoglobulin molecules can be of any type, including, IgG, IgE, IgM, IgD, IgA and IgY, and of any class, including IgG₁, IgG₂, IgG₃, IgG₄, IgA₁ and IgA₂), or of any subclass. Preferably, the monoclonal antibodies for use in the methods and compositions of the invention are IgG antibodies.

The antibodies for use in the methods and compositions of the invention bind to an antigen selected from PA, LF, or EF, or homologs thereof. Preferably, an antibody for use in the methods and compositions of the invention binds with high affinity to the protective antigen (PA) or the lethal factor protein (LF) of B. anthracis, B. cereus, B. thuringiensis, C. perfringens, or a homolog of any of the foregoing.

Affinity is a measure of the strength of binding between an antibody and an antigen. Affinity can be expressed in several ways. One way is in terms of the dissociation constant (K_{d}) of the interaction. K_{d} can be measured by routine methods, include equilibrium dialysis or by directly measuring the rates of antigen-antibody dissociation and association, the k_{off} and kₒₙ rates, respectively (see e.g., Nature, 1993 361:186-87). The ratio of k_{off}/kₒₙ cancels all parameters not related to affinity, and is equal to the dissociation constant K_{d} (see, generally, Davies et al., Annual Rev Biochem, 1990 59:439-473). Thus, a smaller K_{d} means a higher affinity. Another expression of affinity is Kₐ, which is the inverse of K_{d}, or kₒₙ/k_{off}. Thus, a higher Kₐ means a higher affinity. A high affinity antibody for use in the compositions and methods of the invention is an antibody that binds to an antigen of B. anthracis with a K_{d} in the picomolar (pM, 10⁻¹² M) or nanomolar (nM, 10⁻⁹ M) range, or with a Kₐ of at least 10⁷ M⁻¹ or, preferably, from 10⁹ M⁻¹ to 10¹⁰ M⁻¹.

In one embodiment, the antibody binds with a K_{d} of from 1 to 100 pM, from 100 to 250 pM, from 250 to 500 pM, or from 500 to 1000 pM. In another embodiment, the antibody binds with a K_{d} from 1 to 100 nM, from 100 to 250 nM, from 250 to 500 nM, or from 500 to 1000 nM. Preferably, the antibody binds with a K_{d} from 1 to 200 pM or from 1 to 200 nM.

In another embodiment, the antibody binds to the antigen with an affinity constant (Kₐ) of at least 10⁷ M⁻¹, preferably with a Kₐ of from 10⁷ M⁻¹ to 10⁸ M⁻¹, from 10⁸ M⁻¹ to 10⁹ M⁻¹, from 10⁹ M⁻¹ to 10¹⁰ M⁻¹, or from 10¹⁰ M⁻¹ to 10¹¹ M⁻¹. In a preferred embodiment, at least one antibody of the combination binds to its antigen with an affinity of from 10⁹ M⁻¹ to 10¹⁰ M⁻¹.

The monoclonal antibodies useful in the methods and compositions of the invention include chimeric, human, and humanized antibodies, and antigen-binding fragments thereof, which exhibit low toxicity when administered to a subject, preferably a human subject. Toxicity in the context of antibody therapy in a human subject includes, for example, a human anti-murine antibody response (where the antibody is murine) and a human anti-chimeric antibody response (where the antibody is chimeric). Preferably, the antibodies are monoclonal human or humanized antibodies, or antigen-binding fragments thereof.

Antigen-binding fragments of the antibodies include, for example, Fab, Fab′, F(ab′)₂ and Fv fragments. These fragments lack the heavy chain constant fragment (Fc) of an intact antibody and are sometimes preferred because they tend to clear more rapidly from the circulation and have less non-specific binding than an intact antibody. Such fragments are produced from intact antibodies using methods well known in the art, for example by proteolytic cleavage with enzymes such as papain (to produce Fab fragments) or pepsin (to produce F(ab′)₂ fragments). Preferably, an antigen-binding fragment is a dimer of heavy chains (a camelised antibody), a single-chain Fvs (scFv), a disulfide-linked Fvs (sdFv), a Fab fragment, or a F(ab′) fragment.

Preferably, the antibodies for use in the methods and compositions of the invention are monoclonal antibodies. A monoclonal antibody is derived from a substantially homogeneous population of antibodies specific to a particular antigen, which population contains substantially similar epitope binding sites. Such antibodies may be of any immunoglobulin class including IgG, IgM, IgE, IgA, and any subclass thereof. Methods for monoclonal antibody production are well known in the art. Preferably, a monoclonal antibody for use in the methods and compositions of the invention is produced using hybridoma technology.

A human antibody is one in which all of the sequences arise from human genes. Human antibodies include antibodies having the amino acid sequence of a human immunoglobulin and include antibodies isolated from human immunoglobulin libraries or from mice that express antibodies from human genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring, which express human antibodies. The transgenic mice are immunized in the normal fashion with a selected antigen. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, 1995, Int. Rev. Immunol. 13:65-93. For a detailed discussion of this technology for producing human antibodies and human monoclonal antibodies and protocols for producing such antibodies, see e.g., International Publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598. In addition, companies such as Abgenix, Inc. (Freemont, Calif.) and Genpharm (San Jose, Calif.) can be engaged to provide human antibodies directed against a selected antigen using technology similar to that described above.

Human antibodies can also be derived from phage display of human antibody fragments. In phage display methods, functional antibody domains are displayed on the surface of phage particles, which carry the polynucleotide sequences encoding them. In particular, DNA sequences encoding variable heavy and variable light domains are amplified from animal cDNA libraries (e.g., human or murine cDNA libraries of lymphoid tissues). The DNA encoding the variable heavy and variable light domains are recombined together with an scFv linker by PCR and cloned into a phagemid vector. The vector is electroporated in E. coli and the E. coli is infected with helper phage. The phage used in these methods are typically filamentous phage including fd and M13. Phage expressing an antigen binding domain that binds to the antigen epitope of interest can be selected or identified with antigen, e.g., using labeled antigen or antigen bound or captured to a solid surface or bead. Examples of phage display methods include those disclosed in Brinkman et al., 1995, J. Immunol. Methods 182:41-50; Ames et al., 1995, J. Immunol. Methods 184:177; Kettleborough et al., 1994, Eur. J. Immunol. 24:952-958; Persic et al., 1997, Gene 187:9; Burton et al., 1994, Adv. Immunol. 57:191-280; International Application No. PCT/GB91/01134; International Application Publication Nos. WO 90/02809, WO 91/10737, WO 92/01047, WO 92/18619, WO 93/1 1236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108. Preferably, after phage selection, the antibody coding regions from the phage are isolated and used to generate whole antibodies, including human antibodies as described in the above references.

A humanized antibody is an antibody which comprises a framework region having substantially the same amino acid sequence as human receptor immunoglobulin and a complementarity determining region (“CDR”) having substantially the same amino acid sequence as a non-human donor immunoglobulin. A humanized antibody comprises substantially all of at least one, and typically two, variable domains (Fab, Fab′, F(ab′)₂, Fv) in which all or substantially all of the CDR regions correspond to those of the non-human donor immunoglobulin (i.e., the donor antibody) and all or substantially all of the framework regions of the human acceptor immunoglobulin. The acceptor may comprise or consist of a consensus sequence of human immunoglobulins. Preferably, a humanized antibody also comprises at least a portion of an immunoglobulin constant region (Fc), typically that of a human immunoglobulin. Ordinarily, the antibody will contain a light chain and at least the variable domain of a heavy chain. The antibody also may include the CH1, hinge, CH2, CH3, and CH4 regions of the heavy chain. The humanized antibody can be selected from any class of immunoglobulins, including IgM, IgG, IgD, IgA and IgE, and any isotype, including IgG1, IgG2, IgG3 and IgG4. The framework and CDR regions of a humanized antibody need not correspond precisely to the donor and acceptor sequences, e.g., the donor CDR or the acceptor framework may be mutagenized by substitution, insertion or deletion of at least one residue. Such mutations, however, will not be extensive. Usually, at least 75% of the humanized antibody residues will correspond to those of the acceptor framework and donor CDR sequences, more often 90%, and most preferably greater than 95%. A humanized antibody can be produced using variety of techniques known in the art, including but not limited to, CDR-grafting (see e.g., European Patent No. EP 239,400; International Publication No. WO 91/09967; and U.S. Pat. Nos. 5,225,539, 5,530,101, and 5,585,089), veneering or resurfacing (see e.g., European Patent Nos. EP 592,106 and EP 519,596; Padlan, 1991, Mol. Immunol. 28:489-498; Studnicka et al., 1994, Prot. Eng. 7:805-814; and Roguska et al., 1994, Proc. Natl. Acad. Sci. U.S.A. 91:969-973), chain shuffling (see e.g., U.S. Pat. No. 5,565,332), and techniques disclosed in, e.g., U.S. Pat. Nos. 6,407,213, 5,766,886, International Publication No. WO 9317105, Tan et al., 2002, J. Immunol. 169:1119-25, Caldas et al., 2000, Protein Eng. 13:353-60, Morea et al., 2000, Methods 20:267-79, Baca et al., 1997, J. Biol. Chem. 272:10678-84, Roguska et al., 1996, Protein Eng. 9:895-904, Couto et al., 1995, Cancer Res. 55:5973s-5977s, Couto et al., 1995, Cancer Res. 55:1717-22, Sandhu, 1994, Gene 150:409-10, and Pedersen et al., 1994, J. Mol. Biol. 235:959-73. Often, framework residues in the framework regions will be substituted with the corresponding residue from the donor antibody to alter, preferably improve, antigen binding. These framework substitutions are identified by methods well known in the art, e.g by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature 332:323, which are incorporated herein by reference in their entireties).

A chimeric antibody comprises non-human variable region sequences and human constant region sequences. A chimeric antibody may be monovalent, divalent or polyvalent. A monovalent chimeric antibody is a dimer formed by a chimeric heavy chain associated through disulfide bridges with a chimeric light chain. A divalent chimeric antibody is a tetramer formed by two heavy-light chain dimers associated through at least one disulfide bridge. A polyvalent chimeric antibody can also be produced, for example, by employing a heavy chain constant region that aggregates (e.g., from an IgM heavy chain).

A “camelised” antibody is one having a functional antigen binding site comprising only the heavy chain variable domains (VH), rather than the conventional antigen binding site which comprises both the heavy and the light chain variable domains (VL). Preferably, a camelised antibody comprises one or two VH domains and no VL domains. Preferably, a camelised antibody comprises two VH domains. Methods for making camelised antibodies are known in the art. See, for example, Riechmann et al., J. Immunol. Methods, 1999 231:25-38, and U.S. Patent Application Publication Nos. US 2004137570 and US 2004142432.

The antibodies for use in the methods and compositions of the invention may be produced by recombinant expression using techniques known in the art. In one embodiment, the nucleic acid sequences used for recombinant expression are those described in U.S. Patent Application Publication No. 20060258842, published Nov. 16, 2006, and in Albrecht et al., Infection and Immunity 2007 75:5425-5433.

According to the present methods, a combination of at least two antibodies is administered to a subject in need of treatment or prevention of disease caused by B. anthracis, or a bacterium which produces toxins or toxin components homologous to those produced by B. anthracis, or disease caused by the toxins or toxin components themselves. The antibodies of the combination may bind to the same or a different bacterial antigen, however at least two antibodies of the combination bind to a different bacterial antigen. In a preferred embodiment, each of the at least two antibodies binds to a different antigen selected from the protective antigen (PA), lethal factor (LF), and edema factor (EF) of B. anthracis, or a homolog of any of the foregoing.

The antibodies suitable for use in the methods and compositions of the invention are preferably human monoclonal antibodies. Human monoclonal antibodies suitable for use in the claimed methods include the anti-PA and anti-LF antibodies described, for example, in U.S. Patent Application Publication No. 20060258842, published Nov. 16, 2006, and in Albrecht et al., Infection and Immunity 2007 75:5425-5433.

In one embodiment, at least one antibody is an anti-PA antibody which binds to the protective antigen (PA) of B. anthracis, or a homolog thereof, with an affinity (Kₐ) of at least 10⁷ M⁻¹, preferably with a Kₐ of from 10⁷ M⁻¹ to 10⁸ M⁻¹, from 10⁸ M⁻¹ to 10⁹ M⁻¹, from 10⁹ M⁻¹ to 10¹⁰ M⁻¹, or from 10¹⁰ M⁻¹ to 10¹¹ M⁻¹. Preferably, the antibody binds to PA with a Kₐ of from 10⁹ M⁻¹ to 10¹⁰ M⁻¹, or from 10¹⁰ M⁻¹ to 10¹¹ M⁻¹.

In one embodiment, at least one antibody is an anti-EF antibody which binds to the edema factor protein (EF) of B. anthracis, or a homolog thereof, with an affinity (Kₐ) of at least 10⁷ M⁻¹, preferably with a Kₐ of from 10⁷ M⁻¹ to 10⁸ M⁻¹, from 10⁸ M⁻¹ to 10⁹ M⁻¹, from 10⁹ M⁻¹ to 10¹⁰ M⁻¹, or from 10¹⁰ M⁻¹ to 10¹¹ M⁻¹. Preferably, the antibody binds to EF with a Kₐ of from 10⁹ M⁻¹ to 10¹⁰ M⁻¹, or from 10¹⁰ M⁻¹ to 10¹¹ M⁻¹.

In one embodiment, at least one antibody is an anti-LF antibody which binds to the lethal factor protein (LF) of B. anthracis, or a homolog thereof, with an affinity (Kₐ) of at least 10⁷ M⁻¹, preferably with a Kₐ of from 10⁷ M⁻¹ to 10⁸ M⁻¹, from 10⁸ M⁻¹ to 10⁹ M⁻¹, from 10⁹ M⁻¹ to 10¹⁰ M⁻¹, or from 10¹⁰ M⁻¹ to 10¹¹ M⁻¹. Preferably, the antibody binds to LF with a Kₐ of from 10⁹ M⁻¹ to 10¹⁰ M⁻¹, or from 10¹⁰ M⁻¹ to 10¹¹ M⁻¹.

In a specific embodiment, at least two of the antibodies of the combination are the antibodies IQNPA and IQNLF described in U.S. Patent Application Publication No. 20060258842, published Nov. 16, 2006, and in Albrecht et al., Infection and Immunity 2007 75:5425-5433. The IQNPA antibody binds to the B. anthracis protective antigen (PA), specifically to domain IV of the PA protein. The IQNLF antibody binds to B. anthracis lethal factor (LF), specifically to domain I of the LF protein. These antibodies were produced by collecting blood samples from healthy individuals immunized with the United Kingdom-licensed anthrax vaccine following annual booster immunizations. Samples demonstrating anthrax lethal toxin-neutralizing activity in cytotoxicity assays were selected for hybridoma development using a polyethylene glycol-based variant of the hybridoma electrofusion technology described by H. Groen and H. H. Westra (U.S. Patent Application Ser. Nos. 60/710,626 and 11/072,102). Hybridoma fusions were screened for expression of anti-PA- and anti-LF-specific antibodies by enzyme-linked immunosorbent assays (ELISAs). Hybridoma clones producing anti-PA and anti-LF monoclonal antibody IgG were expanded and stabilized, and the antibodies were evaluated for anthrax lethal toxin neutralization. Candidate anti-PA and anti-LF antibodies were isotyped using a human Ig subclass ELISA kit (Invitrogen, Carlsbad, Calif.).

The IQNPA and IQNLF antibodies are produced by stable hybridoma cell lines designated ______ and ______, respectively. The hybridomas ______ and ______, were deposited on ______, pursuant to the requirements of the Budapest Treaty on the International Recognition of the Deposit of Microorganisms for the Purposes of Patent Procedure with the American Type Culture Collection (ATCC), 10801 University Boulevard, Manassas, Va. 20110-2209, under ATCC Designation Nos. ______ and ______, respectively. During the pendency of the subject application, access to the deposit shall be afforded to the Commissioner upon request. All restrictions upon public access to this deposit shall be removed upon the grant of a patent on this application and the deposits shall be replaced if viable samples cannot be made by the depository named hereinabove.

The gamma heavy chain and kappa light chain sequences of the IQNPA and IQNLF antibodies are provided below.

>IQNPA H.gamma. amino acid sequence:
(SEQ ID NO: 1)
MDWIWRILFLVAAATGAHSQVQLVQSGAEVKKPGASVKVSCKASGYTFT
SNAIQWVRQAPGQRLEWVGWINGGDGNTKYSQKFQGRVTISRDISASTA
YMELSSLRSEDTAVYYCARHRLQRGGFDPWGQGTLVTVSSASTKGPSVF
PLAPSSKSTSGGTAALGCLVKDYFPEPVTVSWNSGALTSGVHTFPAVLQ
SSGLYSLSSVVTVPSSSLGTQTYICNVNHKPSNTKVDKRVEPKSCDKTH
TCPPCPAPELLGGPSVFLEPPKPKDTLMISRTPEVTCVVVDVSHEDPEV
KFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQG
NVFSCSVMHEALHNHYTQKSLSLSPGK
>IQNPA L.kappa. amino acid sequence:
(SEQ ID NO: 2)
MEAPAQLLFLLLLWLPDTTGEIVLTQSPGTLSLSPGERATLSCRASQSV
SYSSLAWYQQKPGQAPSLLIYGASSRATGIPDRFSGSGSGPDFTLTISR
LEPEDFAVYYCQHYGNSPYTFGQGTKLEIKRTVAAPSVFIFPPSDEQLK
SGTASVVCLLNNFYPREAKVQWKVDNALQSGNSQESVTEQDSKDSTYSL
SSTLTLSKADYEKHKVYACEVTHQGLSSPVTKSFNRGEC
>IQNLF H.gamma. amino acid sequence:
(SEQ ID NO: 3)
MELGLCWLFLVAILKGVQCEVQLLESGGGLVQPGGSLRLSCSGSGFMFS
SYAMSWVRQAPGKGLEWVSGISGSGGTTNYADSVKGRFTISRDNSKNTL
YMQMNSLRAEDTAVYYCAKDGVYGRLGGSDYWGQGTLVTVSSASTKGPS
VFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDK
THTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQ
QGNVFSCSVMHEGLHNHY-TQK SLSLSPGK
>IQNLF L.kappa. amino acid sequence:
(SEQ ID NO: 4)
MLPSQLIGFLLLWVPASRGEIVLTQSPDFQSVSPKEKVTITCRASQSVG
SSLHWYQQKPDQSPKLLIKYASQSFSGVPSRFSGSGSGTDFTLTINSLE
TEDAATYYCHQSSSLPLTFGGGTKVEIKRTVAAPSVFIFPPSDEQLKSG
TASVVCLLNNFYPREAKVQWKVDNALQSGNSQESVTEQDSKDSTYSLSS
TLTLSKADYEKHKVYACEVTHQGLSSPVTKSF-NRGEC

In one embodiment, at least one antibody of the combination is an anti-PA antibody which neutralizes the protective antigen (PA) and comprises a heavy chain amino acid sequences comprising SEQ ID NO: 1. In another embodiment, the anti-PA antibody comprises a light chain amino acid sequence comprising SEQ ID NO: 2. In a particular embodiment, the anti-PA antibody comprises a heavy chain amino acid sequence comprising SEQ ID NO: 1 and a light chain amino acid sequence comprising SEQ ID NO: 2.

In one embodiment, at least one antibody of the combination is an anti-LF antibody which neutralizes the lethal factor protein (LF) and comprises a heavy chain amino acid sequence comprising SEQ ID NO: 3. In another embodiment, the anti-LF antibody comprises a light chain amino acid sequence comprising SEQ ID NO: 4. In a particular embodiment, In another embodiment, the anti-LF antibody comprises a heavy chain amino acid sequence comprising SEQ ID NO: 3 and a light chain amino acid sequence comprising SEQ ID NO: 4.

In one embodiment, the anti-PA antibody comprises a variable heavy chain domain (VH) having three complementarity determining regions (CDR), each CDR comprising the following amino acid sequence: VH CDR1: KKPGA (SEQ ID NO:5); VH CDR2: SNAIQWVRQAPGQRLEW (SEQ ID NO:6); and VH CDR3: YMELSSLR (SEQ ID NO:7). In another embodiment, the anti-PA antibody comprises a variable light chain domain (VL) having three CDRs, each CDR comprising the following amino acid sequence: VL CDR1: LTQSPGTLSLS (SEQ ID NO:8); VL CDR2: SYSSLAW (SEQ ID NO:9); and VL CDR3: GPDFTLTIS (SEQ ID NO:10). In a particular embodiment, the anti-PA antibody comprises all six of the preceding CDRs.

In one embodiment, the anti-LF antibody comprises a VH domain having three CDRs, each CDR comprising the following amino acid sequence: VH CDR1: VQPGG (SEQ ID NO:11); VH CDR2: SYAMSWVRQAPGKGLEW (SEQ ID NO:12); and VH CDR3: YMQMNSL (SEQ ID NO:13). In another embodiment, the anti-LF antibody comprises a VL domain having three CDRs, each CDR comprising the following amino acid sequence: VL CDR1: TQSPDFQSVSP (SEQ ID NO:14); VL CDR2: SSLHWYQ (SEQ ID NO:15); and VL CDR3: DFTLTINSL (SEQ ID NO:16). In a particular embodiment, the anti-LF antibody comprises all six of the preceding CDRs.

In one embodiment, the anti-PA antibody binds to a protective antigen (PA) polypeptide comprising or consisting of the following amino acid sequence: NNIAVGADES VVKEAHREVI NSSTEGLLLN IDKDIRKILS GYIVEIEDTE GLKEVINDRYDMLNISSLRQ DGKTFIDFKK YNDKLPLYIS NPNYKVNVYA VTKENTIINP SENGDTSTNG IKKILIFSKK GYEIG (SEQ ID NO:17). In another embodiment, the anti-PA antibody binds to a protective antigen (PA) polypeptide comprising or consisting of the following amino acid sequence: TNIYTVLDKI KLNAKMNILI RDKRFHYDRN NIAVGADESV VKEAHREVIN SSTEGLLLNI DKDIRKILSG YIVEIEDTEG LKEVINDRYD MLNISSLRQD GKTFIDFKKY NDKLPLYISN PNYKVNVYAV TKENTIINPS ENGDTSTNGI KKILIFSKKG YEIG (SEQ ID NO:18).

In one embodiment, the anti-PA antibody competitively inhibits the binding of the monoclonal antibody IQNPA to the protective antigen protein of B. anthracis, or a homolog thereof. In another embodiment, the anti-PA antibody competitively inhibits the binding of a polypeptide comprising SEQ ID NO:17 or 18 to the monoclonal antibody IQNPA.

In one embodiment, the anti-LF antibody binds to a lethal factor (LF) polypeptide comprising or consisting of the following amino acid sequence:

(SEQ ID NO: 19)
ERNKTQEEHLK EIMKHIVKIE VKGEEAVKKE AAEKLLEKVP
SDVLEMYKAI GGKIYIVDGD ITKHISLEAL SEDKKKIKDI
YGKDALLHEH YVYAKEGYEP VLVIQSSEDY VENTEKALNV
YYEIGKILSR DILSKINQPY QKFLDVLNTI KNASDSDGQD
LLFTNQLKEH PTDFSVEFLE QNSNEVQEVF AKAFAYYIEP
QHRDVLQLYA PEAFNYMDKF NEQEINLSLE ELKDQ.

In one embodiment, the anti-LF antibody competitively inhibits the binding of the monoclonal antibody IQNLF to the lethal factor protein of B. anthracis, or a homolog thereof. In another embodiment, the anti-LF antibody competitively inhibits binding of the monoclonal antibody IQNLF to a polypeptide comprising SEQ ID NO: 19.

Methods for determining antibody specificity and affinity by competitive inhibition are known in the art, for example, such methods can be found in Harlow, et al., Antibodies: A Laboratory Manual, Cold Spring Harbor Laboratory Press, Cold Spring Harbor, N.Y., 1988; Colligan et al., eds., Current Protocols in Immunology, Greene Publishing Assoc. and Wiley Interscience, N.Y., (1992, 1993); and Muller, Meth. Enzymol. 92:589 601 (1983).

Preferably, the antibodies for use in the methods and compositions of the invention are isolated or purified. An “isolated” or “purified” antibody is substantially free of cellular material or other contaminating proteins from the cell or tissue source from which the antibody is derived, or substantially free of chemical precursors or other chemicals when chemically synthesized. The language “substantially free of cellular material” includes preparations in which the antibody is separated from cellular components of the cells from which it is isolated or recombinantly produced. Thus, antibody that is substantially free of cellular material includes preparations having less than about 30%, or about 20%, or about 10%, or about 5%, or about 1% (by dry weight) of heterologous protein (also referred to herein as a “contaminating protein”). When the antibody is recombinantly produced, it is also preferably substantially free of culture medium, e.g., culture medium represents less than about 20%, or about 10%, or about 5%, or about 1% of the volume of the protein preparation. When the antibody is produced by chemical synthesis, it is preferably substantially free of chemical precursors or other chemicals, e.g., it is separated from chemical precursors or other chemicals that are involved in the synthesis of the protein. Accordingly such preparations of antibody have less than about 30%, or about 20%, or about 10%, or about 5%, or about 1% (by dry weight) of chemical precursors or compounds other than the antibody of interest.

1.1.1 Compositions

The present invention also provides compositions comprising a combination of at least two of the antibodies described above in Section 1.1. Preferably, a composition comprising the antibodies is suitable for administration to a human subject. In one embodiment, the composition is a pharmaceutical composition comprising at least two antibodies, an anti-PA antibody and an anti-LF antibody, and one or more pharmaceutically acceptable carriers or excipients. In one embodiment, the composition is formulated as a liquid. In another embodiment, the composition is lyophilized.

The term excipient broadly refers to a biologically inactive substance used in combination with the active agents, i.e., the antibodies, of the composition. An excipient can be used, for example, as a solubilizing agent, a stabilizing agent, a surfactant, a demulcent, a viscosity agent, a diluent, an inert carrier, a preservative, a binder, a disintegrant, a coating agent, a flavoring agent, or a coloring agent. Preferably, at least one excipient is chosen to provide one or more beneficial physical properties to the composition, such as increased stability and/or solubility of the active agent(s). A “pharmaceutically acceptable” excipient is one that has been approved by a state or federal regulatory agency for use in animals, and preferably for use in humans, or is listed in the U.S. Pharmacopia, the European Pharmacopia or another generally recognized pharmacopia for use in animals, and preferably for use in humans.

Examples of carriers that may be used in the compositions of the present invention include water, mixtures of water and water-miscible solvents, such as C1- to C7-alkanols, vegetable oils or mineral oils comprising from 0.5 to 5% non-toxic water-soluble polymers, natural products, such as gelatin, alginates, pectins, tragacanth, karaya gum, xanthan gum, carrageenin, agar and acacia, starch derivatives, such as starch acetate and hydroxypropyl starch, and also other synthetic products, such as polyvinyl alcohol, polyvinylpyrrolidone, polyvinyl methyl ether, polyethylene oxide, preferably cross-linked polyacrylic acid, such as neutral Carbopol, or mixtures of those polymers. The concentration of the carrier is, typically, from 1 to 100000 times the concentration of the active ingredient.

Further examples of excipients include certain inert proteins such as albumins; hydrophilic polymers such as polyvinylpyrrolidone; amino acids such as aspartic acid (which may alternatively be referred to as aspartate), glutamic acid (which may alternatively be referred to as glutamate), lysine, arginine, glycine, and histidine; fatty acids and phospholipids such as alkyl sulfonates and caprylate; surfactants such as sodium dodecyl sulphate and polysorbate; nonionic surfactants such as such as TWEEN®, PLURONICS®, or a polyethylene glycol (PEG) designated 200, 300, 400, or 600; a Carbowax designated 1000, 1500, 4000, 6000, and 10000; carbohydrates such as glucose, sucrose, mannose, maltose, trehalose, and dextrins, including cyclodextrins; polyols such as mannitol and sorbitol; chelating agents such as EDTA; and salt-forming counter-ions such as sodium.

In one embodiment, the pharmaceutical composition further comprises one or more additional therapeutic agents. In a preferred embodiment, the one or more additional therapeutic agents is selected from an antibiotic, preferably ciprofloxacin or doxycycline.

### 1.2 Methods of Use

The present invention provides methods for the prevention and treatment of disease caused by B. anthracis or a bacterium which produces toxins or toxin components homologous to those produced by B. anthracis, or disease caused by the toxins or toxin components themselves, in a subject in need thereof by administering at least two neutralizing monoclonal antibodies to the subject, each having a different antigen specificity. Each of the at least two antibodies has affinity for a different bacterial antigen selected from the protective antigen (PA), lethal factor (LF), and edema factor (EF) of B. anthracis, or a homolog of any of the foregoing. Preferably, at least one antibody is an anti-protective antigen (PA) antibody. The combination of antibodies is administered to a subject either prophylactically or therapeutically. A subject in need of prophylactic treatment is one who has been exposed to B. anthracis or a bacterium which produces toxins or toxin components homologous to those produced by B. anthracis, or to the toxins or toxin components themselves, but has not developed any clinical signs of infection (post-exposure prophylaxis), or one who is likely to be exposed in the near future (pre-exposure prophylaxis). In one embodiment, prophylactic treatment is administered to a subject who is asymptomatic following exposure. In another embodiment, prophylactic treatment is administered to a subject prior to exposure. In the context of these embodiments, prophylactic treatment results in an inhibition or delay in the onset or progression of at least one clinical symptom associated with the bacterial infection. A subject in need of therapeutic treatment is one who already presents with one or more clinical symptoms of bacterial infection. In one embodiment, therapeutic treatment is administered to a subject following exposure who presents with one or more clinical signs or symptoms of the bacterial infection or toxemia, which may occur in the absence of bacteria.

The invention also provides methods for increasing the survival odds for a subject who has been exposed to B. anthracis or a bacterium which produces toxins or toxin components homologous to the virulence factors produced by B. anthracis or to the toxins or toxin components themselves, in the absence of bacteria, by administering a combination of at least two antibodies to the subject, preferably an anti-PA antibody and an anti-LF antibody. In one embodiment, the antibodies are administered as part of a therapeutic regimen that includes antibiotics, preferably ciprofloxacin and/or doxycycline. Combination therapy with antibiotics is discussed in more detail below in Section 1.2.2.

1.2.1 Administration and Dosages

The antibodies of the present invention can be administered to a subject either separately or together. Preferably, the antibodies are administered at the same time or at substantially the same time. The subject may be any mammal, including, for example, a mouse, a rat, a rabbit, a dog, a pig, a non-human primate, or a human. Preferably, the subject is human. In certain embodiments, one or more of the antibodies is administered in combination with one or more additional therapeutic agents, preferably one or more antibiotics, as described below in Section 1.2.2. The dosage administered will vary depending upon known factors such as the pharmacodynamic characteristics of the particular antibodies, the mode and route of administration, and the age, health, and weight of the subject.

Dosage preferably reflects the total amount of antibody administered to the subject. Exemplary doses include 1 to 20 mg of antibody per kg (mg/kg) of body weight or about 1 to 10 mg/kg of body weight. In a specific embodiment, the total amount of antibody administered is about 2.5 mg/kg, about 5 mg/kg, about 10 mg/kg, about 12 mg/kg, about 14 mg/kg, about 16 mg/kg, about 18 mg/kg, or about 20 mg/kg of the subject's body weight.

The amount of each antibody administered will usually be different, and depends on the efficacy of the particular combination of antibodies. The effective amount of each antibody is determined using routine methods for determining optimal dose and efficacy. Typically, the amount of each antibody administered will be in the range of 1 to 20 mg/kg, preferably 1 to 10 mg/kg, and most preferably 1 to 5 mg/kg body weight. In one embodiment, the antibody combination comprises the IQNPA and IQNLF antibodies, wherein the IQNPA antibody is administered at a dosage of from 1 to 20 mg/kg body weight and the IQNLF antibody is administered at a dosage of from 1 to 10 mg/kg. In a particular embodiment, the IQNPA antibody is administered at a dosage of 10 or 20 mg/kg. Preferably, the total amount of antibody administered is from 1 to 20 mg/kg.

The antibodies for use in the methods of the invention are preferably formulated for intravenous, intramuscular, or subcutaneous administration. In certain embodiments, the antibodies are formulated for administration by injection through another route, such as intradermal or transdermal. In one embodiment, the antibodies are formulated for intravenous administration. However, the antibodies may be formulated for any suitable route of administration.

An effective amount of the antibodies is the amount sufficient to reduce the severity of the disease caused by B. anthracis or a bacterium which produces toxins or toxin components homologous to those produced by B. anthracis, or disease caused by the toxins or toxin components themselves, the amount sufficient to prevent the incidence or advancement of the disease, or the amount sufficient to enhance or improve the therapeutic effect(s) of another therapy or therapeutic agent. Preferably, the effective amount is the amount sufficient to prevent mortality or to expand the treatment window for a subject who has been exposed to B. anthracis or a bacterium which produces toxins or toxin components homologous to those produced by B. anthracis or to the toxins or toxin components themselves, in the absence of bacteria.

The antibodies of the present invention can be administered either as individual therapeutic agents or in combination with other therapeutic agents. The dosage administered will vary depending upon known factors such as the pharmacodynamic characteristics of the particular agent, and its mode and route of administration; age, health, and weight of the recipient; nature and extent of symptoms, kind of concurrent treatment, frequency of treatment, and the effect desired.

Examples of dosing regimens that can be used in the methods of the invention include, but are not limited to, once daily, three times weekly (intermittent), weekly, or every 14 days. In certain embodiments, dosing regimens include, but are not limited to, monthly dosing or dosing every 6-8 weeks. In a preferred embodiment of pre-exposure treatment, the regimen includes dosing once every 2 to 4 weeks. In a preferred embodiment of post-exposure treatment, a single dose is administered as soon as possible following exposure. In one embodiment of post-exposure treatment, the regimen further includes another dose about 2 weeks following exposure. In another embodiment, the regimen includes dosing once a week for 4 to 8 weeks following exposure.

1.2.2 Combination Therapy

In certain embodiments, the antibodies are administered as part of a therapeutic regimen which includes antibacterial agents. Antibacterial agents, including antibiotics, that can be used in combination with the antibodies of the invention include, without limitation, aminoglycoside antibiotics, glycopeptides, amphenicol antibiotics, ansamycin antibiotics, cephalosporins, cephamycins oxazolidinones, penicillins, quinolones, streptogamins, tetracyclins, and analogs thereof. Preferably, the antibody combinations of the invention are administered as part of a therapeutic regimen that includes ciprofloxacin and doxycycline.

In one embodiment, the regimen includes administration of an antibacterial agent at a dosage of from 10-50 mg/kg/day, preferably 20, 25, 30, or 35 mg/kg/day, for 30-90 days, preferably for 60-90 days.

In one embodiment, the antibacterial agent is selected from the group consisting of ampicillin, amoxicillin, ciprofloxacin, gentamycin, kanamycin, neomycin, penicillin G, streptomycin, sulfanilamide, and vancomycin.

In one embodiment, the antibacterial agent is selected from the group consisting of azithromycin, cefonicid, cefotetan, cephalothin, cephamycin, chlortetracycline, clarithromycin, clindamycin, cycloserine, dalfopristin, doxycycline, erythromycin, linezolid, mupirocin, oxytetracycline, quinupristin, rifampin, spectinomycin, and trimethoprim

Additional, non-limiting examples of antibacterial agents for use in combination with the antibodies of the invention include the following: aminoglycoside antibiotics (e.g., apramycin, arbekacin, bambermycins, butirosin, dibekacin, neomycin, neomycin, undecylenate, netilmicin, paromomycin, ribostamycin, sisomicin, and spectinomycin), amphenicol antibiotics (e.g., azidamfenicol, chloramphenicol, florfenicol, and thiamphenicol), ansamycin antibiotics (e.g., rifamide and rifampin), carbacephems (e.g., loracarbef), carbapenems (e.g., biapenem and imipenem), cephalosporins (e.g., cefaclor, cefadroxil, cefamandole, cefatrizine, cefazedone, cefozopran, cefpimizole, cefpiramide, and cefpirome), cephamycins (e.g., cefbuperazone, cefinetazole, and cefminox), folic acid analogs (e.g., trimethoprim), glycopeptides (e.g., vancomycin), lincosamides (e.g., clindamycin, and lincomycin), macrolides (e.g., azithromycin, carbomycin, clarithomycin, dirithromycin, erythromycin, and erythromycin acistrate), monobactams (e.g., aztreonam, carumonam, and tigemonam), nitrofurans (e.g., furaltadone, and furazolium chloride), oxacephems (e.g., flomoxef, and moxalactam), oxazolidinones (e.g., linezolid), penicillins (e.g., amdinocillin, amdinocillin pivoxil, amoxicillin, bacampicillin, benzylpenicillinic acid, benzylpenicillin sodium, epicillin, fenbenicillin, floxacillin, penamccillin, penethamate hydriodide, penicillin o benethamine, penicillin 0, penicillin V, penicillin V benzathine, penicillin V hydrabamine, penimepicycline, and phencihicillin potassium), quinolones and analogs thereof (e.g., cinoxacin, ciprofloxacin, clinafloxacin, flumequine, grepagloxacin, levofloxacin, and moxifloxacin), streptogramins (e.g., quinupristin and dalfopristin), sulfonamides (e.g., acetyl sulfamethoxypyrazine, benzylsulfamide, noprylsulfamide, phthalylsulfacetamide, sulfachrysoidine, and sulfacytine), sulfones (e.g., diathymosulfone, glucosulfone sodium, and solasulfone), and tetracyclines (e.g., apicycline, chlortetracycline, clomocycline, and demeclocycline). Additional examples include cycloserine, mupirocin, tuberin amphomycin, bacitracin, capreomycin, colistin, enduracidin, enviomycin, and 2,4 diaminopyrimidines (e.g., brodimoprim).

### 1.3 Kits

The present invention provides a pharmaceutical pack or kit comprising one or more containers filled with an antibody composition of the invention. In one embodiment, the composition is an aqueous formulation. In one embodiment, the composition is lyophilized. In preferred embodiments, the liquid or lyophilized composition is sterile. In one embodiment, the kit comprises a liquid or lyophilized composition of the invention, in one or more containers, and one or more other prophylactic or therapeutic agents useful for the treatment of a bacterial infection or toxemia. The one or more other prophylactic or therapeutic agents may be in the same container as the antibody composition, or in one or more other containers. Preferably, the one or more other prophylactic or therapeutic agents comprises an antibiotic, preferably ciprofloxacin and/or doxycycline.

In certain embodiments, the kit further comprises instructions for use in the treatment of anthrax (e.g., using the antibody compositions of the invention alone or in combination with another prophylactic or therapeutic agent), as well as side effects and dosage information for one or more routes of administration. Optionally associated with such container(s) is a notice in the form prescribed by a governmental agency regulating the manufacture, use or sale of pharmaceuticals or biological products, which notice reflects approval by the agency of manufacture, use or sale for human administration. While the instructional materials typically comprise written or printed materials they are not limited to such. Any medium capable of storing such instructions and communicating them to an end user is contemplated by this invention. Such media include, but are not limited to, electronic storage media (e.g., magnetic discs, tapes, cartridges, chips), optical media (e.g. CD ROM), and the like. Such media may include addresses to internet sites that provide such instructional materials.

In another embodiment, this invention provides kits for the packaging and/or storage and/or use of the antibody composition described herein, as well as kits for the practice of the methods described herein. The kits can be designed to facilitate one or more aspects of shipping, use, and storage.

All publications and patents mentioned herein are hereby incorporated by reference in their entirety as if each individual publication or patent was specifically and individually indicated to be incorporated by reference.

### EXAMPLES

### 1.4 Pre-Exposure Efficacy

The objective of this study was to examine the ability of two antibodies, IQNPA and IQNLF, when administered prior to exposure, to protect against death due to inhalational anthrax in New Zealand White rabbits. The average aerosol challenge dose for this study was 132±21 LD50s with a range of 99-199 LD50s. Body weight, body temperature, clinical observations, and bacteremia were all examined during the course of this study. The data, discussed in more detail below, demonstrated that both antibodies were able to prolong survival following infection with B. anthracis. Treatment did not affect the weight gain or loss seen in animals following infection, nor was there any significant change in body temperature during the course of infection (data not shown).

1.4.1 Results

1.4.1.1 Survival

Pre-treatment with either IQNLF or IQNPA prolonged survival to 5.8 and 8.12 days, respectively, on average. In the control group, the average time to death was 4.41 days. In addition, both antibodies increased the survival rate following exposure. While no animals survived in the untreated control group, 100% of animals survived for the 14 day study period in the groups receiving either 5 mg/kg or 2.5 mg/kg IQNPA. 75% survival was seen in the groups receiving either 10 mg/kg or 1.25 mg/kg IQNPA as well as in the group receiving 10 mg/ml IQNLF.

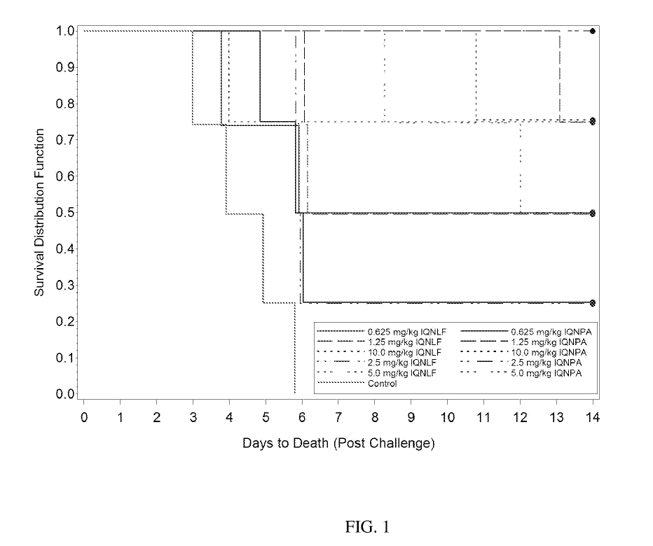

FIG. 1 shows a Kaplan-Meier curve representing time-to-death and survival data for each group. The survival data are tabulated below in Table 1.

TABLE 1
Percentage of animals surviving for 14 days

| | IQNPA % Survival | IQNLF % Survival |
|---|---|---|
| Dose (mg/kg) | (n = 4) | (n = 4) |
| 10 | 75 | 75 |
| 5 | 100 | 50 |
| 2.5 | 100 | 25 |
| 1.25 | 75 | 50 |
| 0.625 | 25 | 50 |

The survival rate of the IQNPA treatment groups receiving 5.0 mg/kg and 2.5 mg/kg was significantly higher than controls by the Fisher's exact test. Due to the small group size (4 animals per group), a statistically significant effect was not observed when the more stringent Bonferroni-Holm adjustment was used.

1.4.1.2 Presence of Bacteria in Blood

The proportion of animals that were bacteremic at any time point post-challenge is given in Table 2 below, along with the 95 percent confidence interval. Twenty-three out of twenty five animals that survived to study day 14 were not bacteremic. Fifteen out of seventeen animals that died or were euthanized prior to study day 14 were bacteremic (by culture). Only five of the animals were bacteremic on study day 2.

25 of the 44 animals (56%) were never positive for bacteria in the blood and 23 of these 25 animals survived through the end of the study. The plated impinger samples were positive for bacterial growth, confirming that the animals were exposed to B. anthracis. The lack of detectable bacteria in the blood may indicate that the circulating levels of antibody inhibited bacterial growth enough to push the amount of bacteria below the level of detection.

Although no statistically significant difference (by Fisher's exact test) was apparent between the control and treatment groups, this result is likely due to the relatively small number of animals (4) per group. The was a statistically significant relationship between death and bacteremia (approximately 88 percent of animals that were bacteremic at any time point died).

TABLE 2
Proportion of animals bacteremic

| | | | | | One-sided Fisher's Exact | |
|---|---|---|---|---|---|---|
| | | | | Proportion | P-value, Comparison | |
| | Treatment | | # | Bacteremic | to Group 6 | |
| | Test | Dose | Bacteremic/ | (95% Confidence | | Bonferroni- |
| Group | Material | mg/kg | Total | Interval) | Unadjusted | Holm Adjusted |
| 1 | IQNPA | 10.0 | 3/4 | 0.75 (0.01, 0.81) | 0.7857 | 1.0000 |
| 2 | IQNPA | 5.0 | 0/4 | 0.00 (0.40, 1.00) | 0.0714 | 0.7143 |
| 3 | IQNPA | 2.5 | 0/4 | 0.00 (0.40, 1.00) | 0.0714 | 0.7143 |
| 4 | IQNPA | 1.25 | 0/4 | 0.00 (0.40, 1.00) | 0.0714 | 0.7143 |
| 5 | IQNPA | 0.625 | 3/4 | 0.75 (0.01, 0.81) | 0.7857 | 1.0000 |
| 6 | Control | 1.0 ml/kg | 3/3^{¥} | 1.00 (0.29, 1.00) | | |
| 7 | IQNLF | 10.0 | 1/4 | 0.25 (0.19, 0.99) | 0.2429 | 1.0000 |
| 8 | IQNLF | 5.0 | 1/4 | 0.25 (0.19, 0.99) | 0.2429 | 1.0000 |
| 9 | IQNLF | 2.5 | 2/3^{±} | 0.67 (0.01, 0.91) | 0.5000 | 1.0000 |
| 10 | IQNLF | 1.25 | 2/4 | 0.50 (0.07, 0.93) | 0.5000 | 1.0000 |
| 11 | IQNLF | 0.625 | 2/4 | 0.50 (0.07, 0.93) | 0.5000 | 1.0000 |

^{¥}Bacteremia sample could not be processed for one animal because sample was lost due to broken tube. Therefore, there were only three animals in this group where presence of bacteremia could be determined at all time points.
^{±}Bacteremia sample could not be drawn for animal K94338. Therefore, there were only three animals in this group where presence of bacteremia could be determined at all time points.

1.4.1.3 Pharmacokinetic Profiles

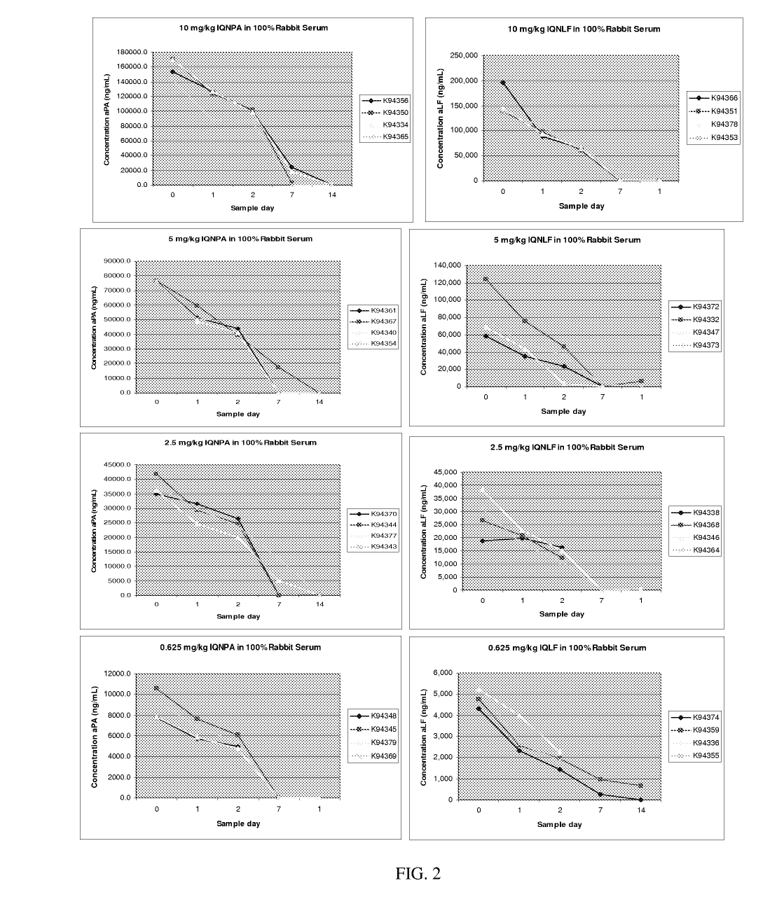

Sera drawn from the animals were frozen and shipped to IQ Corp. for pharmacokinetic analysis. At IQ Corporation, the sera were defrosted and subjected to a quantitative ELISA for IQNPA and IQNLF. Sera from the control rabbits scored below the lower level of quantification of the ELISA. The pharmacokinetic profiles of the groups injected with (10, 5, 2.5 and 0.625 mg/kg) IQNPA or IQNLF are plotted in the FIG. 2. The profiles show a normal decrease of antibody concentrations in time. The mean half-life of the IQNPA antibody was 61.4 hrs.+/−11 hrs; the mean half-life of the IQNLF antibody was 39.5 hrs+/−5.2 hrs.

1.4.1.4 Clinical Observations

Clinical observations were taken from day 0 through day 14 or time of death. All animals were in good health prior to challenge. Lethargy, stool abnormalities (soft stool, diarrhea, and no stool) and lack of eating were the most common clinical observations noted post-challenge. Sixty five percent (13/20) of rabbits receiving IQNPA demonstrated abnormal clinical observations including diarrhea, not eating and lethargy. Seventy-five percent (15/20) of rabbits receiving IQNLF presented with some form of clinical manifestation including diarrhea, not eating, soft stool, and lethargy. Three quarters of the control animals presented with some form of clinical manifestation including not eating and lethargy.

1.4.2 Methods

Study Design: All rabbit studies were performed at Battelle Biomedical Research Center, located at State Route 142, West Jefferson, Ohio 43162. Forty-four (22 male and 22 female) specified pathogen free New Zealand white rabbits (purchased from Covance Laboratories) weighing between 2.0-4.0 kg at the time of randomization were placed in the study. Four additional rabbits were housed as extras until the end of the study. All animals were free of malformations and clinical symptoms of disease prior to placement on study. Prior to treatment, rabbits were assigned to one of eleven groups (four rabbits per group), one of two aerosol challenge days (two rabbits per group per day), and a challenge order per day. Randomization occurred based on animal weights.

Pre-Exposure Dosing: Approximately twenty-four hours prior to challenge, rabbits were intravenously administered one of two antibodies at doses of 10.0, 5.0, 2.5, 1.25, or 0.625 mg/kg IQNPA or 10.0, 5.0, 2.5, 1.25, or 0.625 IQNLF (see Table 1, supra). Groups 1-5 received the IQNPA antibody at doses ranging from 10.0 mg/kg to 0.625 mg/kg. Animals in group 6 received buffer only as a control. Groups 7-11 received the IQNLF antibody at doses ranging from 10.0 mg/kg to 0.625 mg/kg. All four rabbits in each group received the indicated doses. Administration of the appropriate dose of either IQNPA or IQNLF was verified by documentation kept during the process of administering.

Aerosol Challenge: This study required two aerosol challenge days with 22 rabbits challenged per day. The overall average dose for the two study days was 132 LD50s with an average challenge dose of 133±23 LD50s for the first day and 131±19 LD50s for the second day. The mass-median aerodynamic diameter for challenge material aerosols on day one was 1.17 μm and the mass-median aerodynamic diameter for challenge material aerosols on day two was 1.18 μm. Rabbits were transported into the BL-3 facility 6 days prior to challenge to allow time for acclimation. On Study Day 0, rabbits were placed into a plethysmography chamber and passed into a Class III cabinet system, and aerosol challenged with a targeted dose of 100 LD50s B. anthracis (Ames strain) spores aerosolized by a Collision nebulizer. Aerosol concentrations of B. anthracis were quantified by determination of cfu. Effluent streams were collected directly from an animal exposure port by an in-line impinger (Model 7541, Ace Glass Incorporated). Serial dilutions of impinger samples were plated and enumerated for challenge dose assessment.

Blood Collection: Blood was drawn from the medial auricular artery or the marginal ear vein. Oil of wintergreen (topical) or acepromazine (1-5 mg/kg) subcutaneously) was used to facilitate blood sampling via the ear. Amounts of blood collected were within the guidelines established by the Battelle IACUC, derived in part from the Canadian Guide to the Care and Use of Experimental Animals.

Bacteremia (Culture): Blood collected in EDTA tubes on study days 0, 2, 14 and/or time of death were cultured, by streaking ˜40 μl of whole blood over blood agar plates, to determine the presence or absence of B. anthracis.

Clinical Observations: Animals were monitored twice daily for abnormal clinical signs (such as respiratory distress, inappetence, inactivity, seizures and moribundity) until Study Day 14. On study day 13, AM observations were not recorded for four of the rabbits. Any rabbits that were moribund, as assessed by a highly trained life sciences technician, Battelle veterinarian, or Study Director, were euthanized.

Sera Collection and Shipment: Approximately 2.0 ml of whole blood was collected into SST tubes on the days 0, 1, 2, 7, 14 and time of death. This blood was processed and the serum collected. Serum was then filtered, and checked for sterility for shipment to IQ Corporation for serological analysis. When possible, a terminal sample was taken from any animal found dead or found to be moribund prior to euthanasia.

### 1.5 Post-Exposure Efficacy

### Experiment 1

The objective of this study was to examine the ability of two antibodies, IQNPA and IQNLF, when administered after exposure to B. anthracis, either alone or in combination, to protect against death due to inhalational anthrax in rabbits. In rabbits, a body temperature increase of about 2 degrees Fahrenheit is observed at the start of the symptomatic period, which occurs about 25-29 hours post-exposure. Thus, in the following three experiments, treatment during the period of time from 0 to 24 hours following exposure is considered prophylactic treatment. Treatment after 32 hours is considered therapeutic treatment.

The average aerosol challenge dose for this study was 132±30 LD50s, with an average challenge dose of 144±28 LD50s for the first day of challenges and an average dose of 119±31 LD50s for the second day of challenges. Body weight, body temperature, clinical observations and bacteremia were all examined during the course of this study. The data, discussed in more detail below, demonstrate that both the IQNPA and IQNLF antibodies increase survival when administered 24 hours following exposure to inhalational anthrax. The data further demonstrate that the combination of both antibodies resulted in an increased probability of survival compared to either antibody administered alone.

1.5.1 Results

1.5.1.1 Survival

In general, animals treated with either the IQNPA or IQNLF antibodies, or their combination, survived longer than untreated controls. The average time to death for the control animals was 3.87 days. The average time to death in the IQNLF treatment group was 4.72 days; whereas the average time to death in the IQNPA treatment group was 5.52 days. The average time to death for animals receiving a combination of antibodies was 4.53, 3.24, and 6.06 days for treatment groups having doses of IQNPA+IQNLF of 1.25+3.75, 0.625+1.88, and 0.3125 mg/kg+0.94 mg/kg.

The highest dose of IQNPA, 5.0 mg/kg, resulted in 100% survival. The lower IQNPA doses, 2.5 mg/kg and 1.25 mg/kg, resulted in 50% and 33% survival, respectively. The two highest doses of IQNLF (15 mg/kg and 7.5 mg/kg) resulted in 66% survival and the lowest dose (3.75 mg/kg) resulted in 33% survival. None of the animals in the control group survived. Table 3 shows the survival data and results of the Fisher's Exact Test for each treatment group.

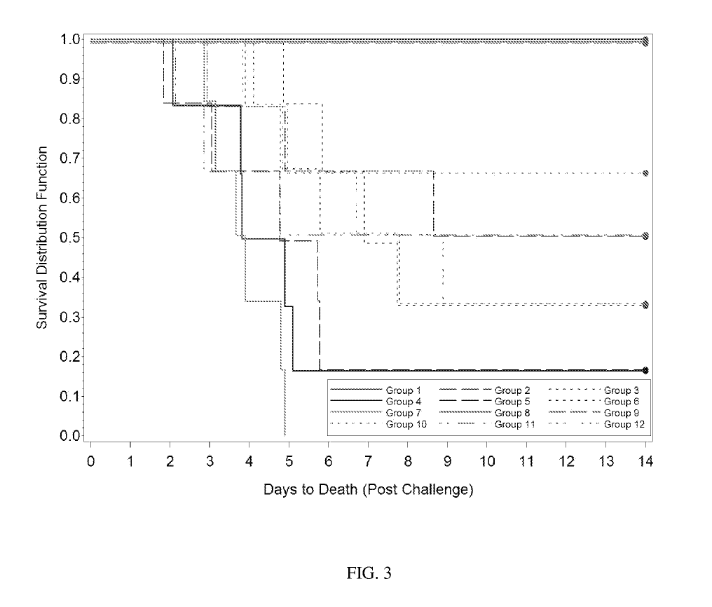

The data further show that when administered in combination, the two antibodies are capable of working in a coordinated manner to eradicate infection. Importantly, 50% survival was observed in the Group 11 even at doses as low as 0.625 mg/kg IQNPA and 1.88 mg/kg IQNLF. The data also show that survival is dose-dependent. FIG. 3 shows the Kaplan-Meier curves for each group.

TABLE 3
Survival Data

| | | | | | One-sided Fisher's Exact | |
|---|---|---|---|---|---|---|
| | | | | | P-value, Comparison to the | |
| | Treatment | | # | Survival Rate | Control Group (Group 7) | |
| | Test | Dose | Survived/ | (95% Confidence | | Bonferroni- |
| Group | Material | (mg/kg) | Total | Interval) | Unadjusted | Holm Adjusted |
| 1 | IQNPA | 5.0 | 6/6 | 1.00 (0.54, 1.00) | 0.0011* | 0.0119* |
| 2 | IQNPA | 2.5 | 3/6 | 0.50 (0.12, 0.88) | 0.0909 | 0.6364 |
| 3 | IQNPA | 1.25 | 2/6 | 0.33 (0.04, 0.78) | 0.2273 | 1.0000 |
| 4 | IQNLF | 15 | 1/6 | 0.17 (0.00, 0.64) | 0.5000 | 1.0000 |
| 5 | IQNLF | 7.5 | 1/6 | 0.17 (0.00, 0.64) | 0.5000 | 1.0000 |
| 6 | IQNLF | 3.75 | 2/6 | 0.33 (0.04, 0.78) | 0.2273 | 1.0000 |
| 7 | Control | PBS Alone | 0/6 | 0.00 (0.00, 0.46) | | |
| 8 | IQNPA + IQNLF | 5.0 + 15 | 6/6 | 1.00 (0.54, 1.00) | 0.0011* | 0.0119* |
| 9 | IQNPA + IQNLF | 2.5 + 7.5 | 6/6 | 1.00 (0.54, 1.00) | 0.0011* | 0.0119* |
| 10 | IQNPA + IQNLF | 1.25 + 3.75 | 4/6 | 0.67 (0.22, 0.96) | 0.0303* | 0.2424 |
| 11 | IQNPA + IQNLF | 0.625 + 1.88 | 3/6 | 0.50 (0.12, 0.88) | 0.0909 | 0.6364 |
| 12 | IQNPA + IQNLF | 0.3125 + 0.94 | 2/6 | 0.33 (0.04, 0.78) | 0.2273 | 1.0000 |

TABLE 4
Percentage Survival

| | | | Percent |
|---|---|---|---|
| Group | Treatment | Dose (mg/kg) | Survival |
| 1 | IQNPA | 5.00 | 100 |
| 2 | IQNPA | 2.50 | 50 |
| 3 | IQNPA | 1.25 | 33 |
| 4 | IQNLF | 15.00 | 16 |
| 5 | IQNLF | 7.50 | 16 |
| 6 | IQNLF | 3.75 | 33 |
| 7 | Control | 1 mL/kg | 0 |
| 8 | IQNPA + IQNLF | 5.0 + 15.0 | 100 |
| 9 | IQNPA + IQNLF | 2.5 + 7.5 | 100 |
| 10 | IQNPA + IQNLF | 1.25 + 3.75 | 66 |
| 11 | IQNPA + IQNLF | 0.625 + 1.88 | 50 |
| 12 | IQNPA + IQNLF | 0.3125 + 0.94 | 33 |

Statistical analysis showed a significant difference for Groups 1, 8, and 9 when compared to the control group using a Bonferroni Holm adjustment to control the overall level of significance at 0.05. When total antibody dose was modeled against the probability of survival, the protection provided by the IQNPA antibody alone was not significantly different from that of the combined antibodies. However, the protection provided by the IQNLF antibody alone was significantly less than that provided by either the IQNPA alone or the combined antibodies. When the dose of IQNPA or IQNLF antibody alone was modeled separately against the probability of survival, the combined antibodies provided significantly greater protection than either of the single antibodies. The odds of survival for animals treated with the combined antibodies were about 9.6 times higher than for the IQNPA antibody alone and about 25.6 times higher than for the IQNLF antibody alone.

1.5.1.2 Presence of Bacteria in Blood

Table 5 shows the proportion of animals that were bacteremic at any time point during the 14 day study period with a 95 percent binomial confidence interval. About half (51% or 37/72) of the challenged animals were bacteremic on day 1. All but three of the animals that died or were euthanized prior to study day 14 were bacteremic. The majority of the surviving animals were not bacteremic on day 14 as determined by blood culture. Of these surviving animals, 33% (12/36) were bacteremic at some time point.

For Group 1, which received 5.0 mg/kg IQNPA, 3/6 animals were bacteremic on study days 1 and 2, but negative at the end of study (day 14). The other three animals in this group were not bacteremic at any time point. These data indicate that, at least in these animals, the IQNPA antibody was able to suppress bacterial proliferation. The fact that three of the animals in this group were not found to be bacteremic does not mean that they were not exposed to B. anthracis. Exposure was confirmed by the plate counts for the impinger samples taken during the exposure process. It is possible that IQNPA has completely cleared the infection when given 24 after exposure.

For Groups 8 and 9, all surviving animals were bacteremia negative at the end of the study. For Group 8, 83% (5/6) of the animals were not bacteremic at any time point. For Group 9, 66% (4/6) of the animals were not bacteremic at any time point. As discussed above, these data indicate that the antibodies were able to suppress bacterial proliferation. Many of the animals receiving lower doses of the antibodies were not only bacteremic on study day 1, but also at the time of euthanasia (or when found dead) prior to day 14.

TABLE 5
Incidence of Bacteremia

| | | | | | One-sided Fisher's Exact | |
|---|---|---|---|---|---|---|
| | | | | Proportion | P-value, Comparison to the | |
| | Treatment | | # | Bacteremic | Control Group (Group 7) | |
| | Test | Dose | Bacteremic/ | (95% Confidence | | Bonferroni- |
| Group | Material | (mg/kg) | Total | Interval) | Unadjusted | Holm Adjusted |
| 1 | IQNPA | 5.0 | 3/6 | 0.50 (0.12, 0.88) | 0.0909 | 0.8182 |
| 2 | IQNPA | 2.5 | 3/6 | 0.50 (0.12, 0.88) | 0.0909 | 0.8182 |
| 3 | IQNPA | 1.25 | 5/6 | 0.83 (0.36, 1.00) | 0.5000 | 1.0000 |
| 4 | IQNLF | 15 | 5/6 | 0.83 (0.36, 1.00) | 0.5000 | 1.0000 |
| 5 | IQNLF | 7.5 | 5/6 | 0.83 (0.36, 1.00) | 0.5000 | 1.0000 |
| 6 | IQNLF | 3.75 | 5/6 | 0.83 (0.36, 1.00) | 0.5000 | 1.0000 |
| 7 | Control | PBS Alone | 6/6 | 1.00 (0.54, 1.00) | | |
| 8 | IQNPA + IQNLF | 5.0 + 15 | 1/6 | 0.17 (0.00, 0.64) | 0.0076* | 0.0833 |
| 9 | IQNPA + IQNLF | 2.5 + 7.5 | 2/6 | 0.33 (0.04, 0.78) | 0.0303* | 0.3030 |
| 10 | IQNPA + IQNLF | 1.25 + 3.75 | 4/6 | 0.67 (0.22, 0.96) | 0.2273 | 1.0000 |
| 11 | IQNPA + IQNLF | 0.625 + 1.88 | 3/6 | 0.50 (0.12, 0.88) | 0.0909 | 0.8182 |
| 12 | IQNPA + IQNLF | 0.3125 + 0.94 | 5/6 | 0.83 (0.36, 1.00) | 0.5000 | 1.0000 |

Since the level of bacteria in the blood was often undetectable, a statistically significant correlation between bacteremia and treatment could not be determined. However, there was sufficient evidence (p value<0.0001) to conclude that whether an animal was bacteremic at any time point was related to whether the animal died. Approximately 72 percent of animals that were bacteremic at any time point died and 94 percent of animals that died were bacteremic at some point.

1.5.1.3 Clinical Observations

Clinical observations were done from day 0 through day 14 or at time of death. Lethargy, stool abnormalities (soft stool, diarrhea, and no stool), and lack of eating were the most common clinical observations noted during the post-challenge observation period. One hundred percent of the control animals displayed clinical symptoms including not eating and lethargy from day 2 post-challenge until death. In Group 1 (5 mg/kg IQNPA), 66% (4/6) of animals displayed clinical symptoms for 6/14 days of the post-challenge period. In Group 2 (2.5 mg/kg IQNPA), 83% (5/6) of animals displayed clinical symptoms for 8/14 days. In Group 3 (1.25 mg/kg IQNPA), 100% (6/6) of animals displayed clinical symptoms on 9/14 days. In Group 8 (5.0 IQNPA+15.0 IQNLF), 50% (3/6) of animals displayed clinical symptoms on 5/14 days. In Group 9 (2.5 IQNPA+7.5 IQNLF), 66% (4/6) of animals displayed clinical symptoms on 5/14 days. In Group 10 (1.25 IQNPA+3.75 IQNLF), 66% (4/6) of animals displayed clinical symptoms on 9/14 days. In Group 11 (0.625 IQNPA+1.88 IQNLF), 83% (5/6) of animals displayed clinical signs on 9/14 days. In Group 12 (0.3125 IQNPA+0.94 IQNLF), 83% (5/6) of animals displayed clinical signs on 9/14 days. These data indicate that all animals have gone through a symptomatic period.

1.5.2 Methods

Test System: Seventy-two (36 male and 36 female) specific pathogen free New Zealand white rabbits (purchased from Covance Laboratories) weighing between 2.0 to 4.0 kg at the time of randomization that were in good health were placed on study. Six additional rabbits were housed as extras until the completion of the study. Prior to challenge, rabbits were assigned to one of twelve groups (six rabbits per group) based on animal weights, one of three aerosol challenge days (two rabbits per group per day) and a challenge order per day. Randomization was based on animal weights. The study was continued for 14 days.

Aerosol Challenge: This study required two aerosol challenge days with 36 rabbits challenged per day. Rabbits were transported into the BL-3 facility 6 days prior to challenge to allow time for acclimation. On Study Day 0, rabbits were placed into a plethysmography chamber and passed into a Class III cabinet system, and aerosol challenged with a targeted dose of 100 LD50s B. anthracis (Ames strain) spores aerosolized by a Collision nebulizer. Aerosol concentrations of B. anthracis were quantified by determination of cfu. effluent streams collected directly from an animal exposure port by an in-line impinger (Model 7541, Ace Glass Incorporated). Serial dilutions of impinger samples were plated and enumerated. The overall average dose for the two study days was 132±30 LD50s with an average challenge dose of 144±28 LD50s for the first day of challenges and an average dose of 119±31 LD50s for the second day of challenges. The mass-median aerodynamic diameter for challenge material aerosols on day one was 1.14 μm and the mass-median aerodynamic diameter for challenge material aerosols on day two was 1.13 μm (as determined with an Aerodynamic Particle Sizer (APS model 3321, TSI Inc, St. Paul, Minn.).

Post-Exposure Dosing: Approximately twenty-four hours post-challenge with B. anthracis, the rabbits were administered one of two antibodies at varying doses, or in combination (see Table 3, supra). Groups 1-3 received IQNPA at doses ranging from 5 mg/kg to 1.25 mg/kg. Groups 4-6 received IQNLF at doses ranging from 15.0 mg/kg to 3.75 mg/kg. Group 7 received buffer only as a control. Groups 8-12 received decreasing doses of the combination treatment (IQNPA+IQNLF). All six rabbits in each group received the indicated doses as indicated by signed paperwork filled out during the dosing process. All treatments were via a single bolus dose.

Blood Collection: Blood samples were collected on days −1, 1 (prior to treatment), 2 and 14, or at time of death. Blood was drawn from the marginal ear vein according. Oil of wintergreen (topical) or acepromazine (1-5 mg/kg subcutaneously) was utilized to facilitate blood sampling via the ear. Amounts of blood collected fell within the guidelines established by the Battelle IACUC, derived in part from the Canadian Guide to the Care and Use of Experimental Animals.

Bacteremia (Culture): Blood collected in EDTA tubes on study days −1, 1, 2, 14 and/or time of death were cultured by streaking ˜40 μl of whole blood over blood agar plates, to determine the presence or absence of B. anthracis bacteremia.

Sera Collection and Shipment: Approximately 2.0 ml of whole blood was collected into SST tubes. This blood was processed and the serum collected. Serum was then filtered, and checked for sterility for shipment to IQ Corporation for serological analysis. When possible, a terminal sample was taken from any animal found dead or found to be moribund prior to euthanasia.

Clinical Observations: Animals were monitored twice daily by laboratory animal personnel near the beginning and end of each workday for abnormal clinical signs (such as respiratory distress, inappetence, inactivity, seizures and moribundity) until Study Day 14. Any rabbits that were moribund, as assessed by a highly trained life sciences technician, Battelle veterinarian, or Study Director, were euthanized.

### 1.6 Post-Exposure Efficacy

### Experiment 2

The main objective of this study was to further examine the efficacy of the combined treatment of IQNLF and IQNPA against inhalational anthrax infection. The target infectious dose for this study was 100 LD50s; the average aerosol challenge dose for the study was 91±27 with a range of 47-149 LD50s. The log-rank test applied to the time-to-death data showed that the pooled control group was significantly less protected than groups treated with combined antibodies when time to death was considered in addition to the overall survival rates. Overall, IQNLF alone did not provide as high a level of protection as the combined treatment.

1.6.1 Results

1.6.1.1 Survival

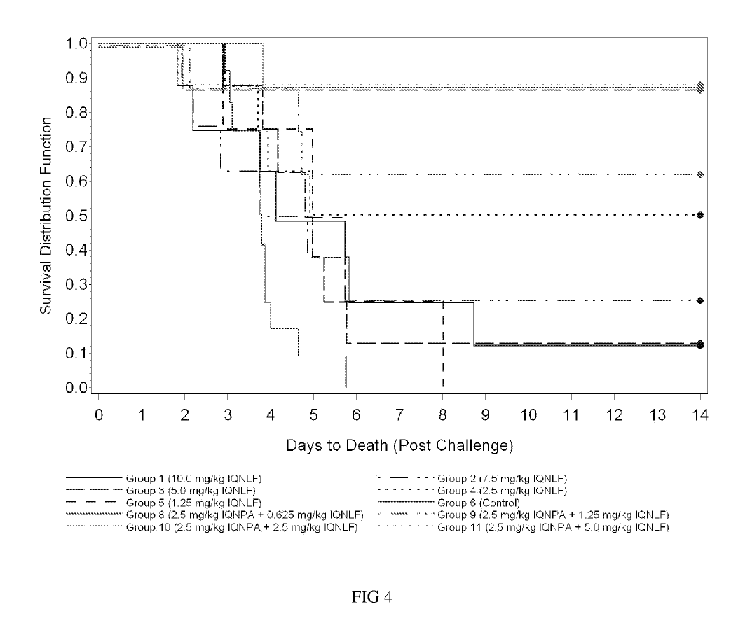

Fifty-seven percent (46/80, regardless of treatment) of the challenged animals succumbed to the infection, with an average time to death of approximately 4.08 days. For the control group, 100% (12/12) of animals succumbed to infection with an average time to death of 3.8 days. For Groups 1-5 (IQNLF alone at 10.00, 7.50, 5.00, 2.50, and 1.25 mg/kg, respectively), 87.5% (7/8), 75% (6/8), 87.5% (7/8), 50% (4/8), and 100% (4/4) of the animals succumbed to disease with an average time to death of 4.6, 3.5, 4.5, 3.8, and 5.39 days respectively. For Groups 8-11 (IQNPA+IQNLF in combination at 2.5+0.625, 2.5+1.25, 2.5+2.5, and 2.5+5.0 mg/kg respectively), 25% (2/8), 38% (3/8), 12.5% (1/8), and 0% (0/8) of the animals succumbed to disease. FIG. 4 is a Kaplan-Meier curve representing time-to-death and survival data for each group.

Table 6 summarizes the survival data for each group. Confidence intervals for the survival rates and the results of Fisher's exact test comparisons of survival rates to the control group are also provided. According to the unadjusted p values from Fisher's exact test, treatment group 4 (IQNLF, 2.5 mg/kg) as well as groups 8 through 11 (all groups with combined antibody doses of IQNPA and IQNLF) had significantly higher survival rates than the pooled control group (group 6). When a Bonferroni Holm adjustment was used to control the overall level of significance at 0.05, the same groups (4 and 8 through 11) had a significantly higher survival rate than the control group.

TABLE 6
Survival Rates

| | | | | | One-sided Fisher's Exact | |
|---|---|---|---|---|---|---|
| | | | | | P-value, Comparison | |
| | Treatment | | No. | Survival Rate | to Group 6 | |
| | Test | Dose | Survived/ | (95% Confidence | | Bonferroni- |
| Group | Material | (mg/kg) | Total | Interval) | Unadjusted | Holm Adjusted |
| 1 | IQNLF | 10.0 | 1/8 | 0.13 (0.00, 0.53) | 0.2000 | 0.6000 |
| 2 | IQNLF | 7.5 | 2/8 | 0.25 (0.03, 0.65) | 0.0737 | 0.2947 |
| 3 | IQNLF | 5.0 | 1/8 | 0.13 (0.00, 0.53) | 0.2000 | 0.6000 |
| 4 | IQNLF | 2.5 | 4/8 | 0.50 (0.16, 0.84) | 0.0072* | 0.0361* |
| 5 | IQNLF | 1.25 | 0/4 | 0.00 (0.00, 0.60) | 0.5000 | 0.6000 |
| 6 | Control | PBS 1 mL/kg | 0/12 | 0.00 (0.00, 0.26) | | |
| 8 | IQNPA + IQNLF | 2.5 + 0.625* | 7/8 | 0.88 (0.47, 1.00) | <0.0001* | 0.0005* |
| 9 | IQNPA + IQNLF | 2.5 + 1.25* | 5/8 | 0.63 (0.24, 0.91) | 0.0018* | 0.0108* |
| 10 | IQNPA + IQNLF | 2.5 + 2.5* | 7/8 | 0.88 (0.47, 1.00) | <0.0001* | 0.0005* |
| 11 | IQNPA + IQNLF | 2.5 + 5.0* | 7/8 | 0.88 (0.47, 1.00) | <0.0001* | 0.0005* |

TABLE 7
Percent Survival

| | | Treatment | | |
|---|---|---|---|---|
| | | Time (Hrs post- | | Percent |
| Group | Treatment | challenge) | Dose (mg/kg) | Survival |
| 1 | IQNLF | 24 | 10.00 | 12.5 |
| 2 | IQNLF | 24 | 7.50 | 25 |
| 3 | IQNLF | 24 | 5.00 | 12.5 |
| 4 | IQNLF | 24 | 2.50 | 50 |
| 5 | IQNLF | 24 | 1.25 | 0 |
| 6 | Control | 24 | 1.0 mL/kg PBS | 0 |
| 8 | IQNPA + IQNLF | 24 | 2.5 + 0.625 | 87.5 |
| 9 | IQNPA + IQNLF | 24 | 2.5 + 1.25 | 62.5 |
| 10 | IQNPA + IQNLF | 24 | 2.5 + 2.5 | 87.5 |
| 11 | IQNPA + IQNLF | 24 | 2.5 + 5.0 | 87.5 |

Table 8 shows the estimates and p values for the effects included in the final logistic regression model that models survival with effects for the base 10 log transformed combined antibody dose and treatment (IQNLF or IQNPA+IQNLF). The interaction between dose and treatment was not significant (p value=0.5023) and so was not included in the final model. The effect for the log transformed dose was not statistically significant (p value=0.8460). Thus, there was not a statistically significant relationship between treatment dose and probability of survival. However, the overall effect for treatment was statistically significant at the 0.05 level (p value<0.0001) indicating that survival rates differed among the two treatments (IQNLF and combined IQNPA+IQNLF). Table 9 shows the odds ratios for the model.

TABLE 8
Effects included in Logistic Regression Model Fitted to Combined
Antibody Dose and Treatment (IQNLF or IQNLF + IQNLP)

| Effect | P-value |
|---|---|
| Intercept | 0.7604 |
| Treatment | <0.0001* |
| Log₁₀(Dose) | 0.8460 |

TABLE 9
Summary of Odds Ratios for Logistic Regression Model
Fitted to Combined Antibody Dose and Treatment

| Treatment Group Comparison | Odds Ratio | P-Value |
|---|---|---|
| (IQNLF + IQNPA) vs. IQNLF | 15.15 | <0.0001* |
| Log₁₀(Dose) | 0.79 | 0.8460 |

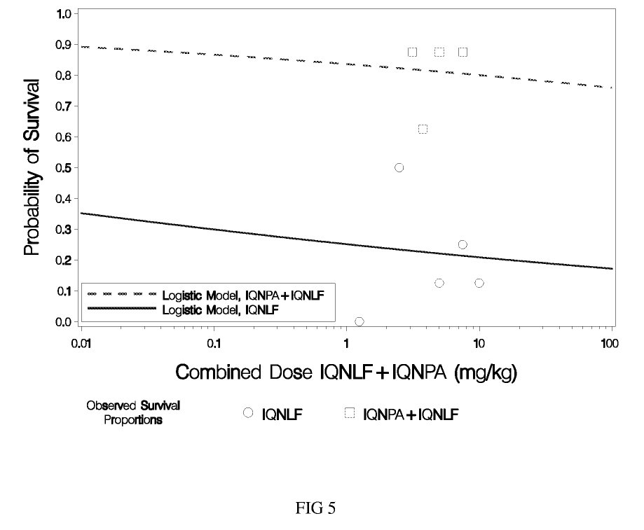

The data show that the odds of survival for animals treated with both antibodies (IQNPA+IQNLF) are about 15 times higher than for animals treated with the IQNLF antibody alone. FIG. 5 plots the estimated logistic regression curves for each treatment along with points showing the proportion of animals that survived for each dose group and treatment.

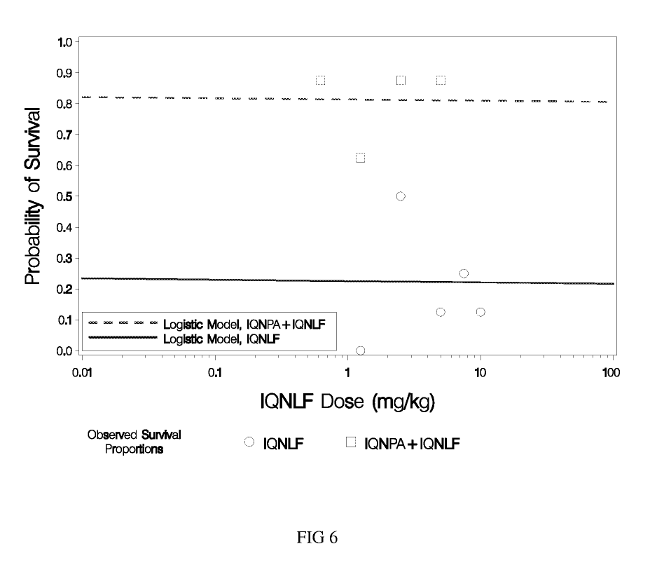

Table 10 shows the estimates and p values for the effects included in the logistic regression model fitted to the base 10 log transformed IQNLF dose and an indicator for treatment (IQNLF or IQNPA+IQNLF). The interaction between IQNLF dose and treatment was not significant (p value=0.5480) and so was not included in the final model. The effect for the log transformed IQNLF dose was not statistically significant (p value=0.9788). The treatment effect was statistically significant at the 0.05 level (p value=0.0002) indicating that survival rates differed among the two treatments (IQNLF and combined IQNPA+IQNLF). Table 11 presents the odds ratios from the model. The odds of survival for animals treated with both antibodies (IQNPA+IQNLF) are about 15 times higher for animals treated with IQNLF alone. FIG. 6 plots the estimated logistic regression curves for the IQNLF and combined treatments along with points showing the proportion of animals that survived for each group.

TABLE 10
Effects included in Logistic Regression
Model Fitted to IQNLF Dose and Treatment

| Effect | P-value |
|---|---|
| Intercept | 0.8243 |
| Treatment | 0.0002* |
| Log₁₀(IQNLF Dose) | 0.9788 |

TABLE 11
Summary of Odds Ratios for Logistic Regression
Model Fitted to IQNLF Dose and Treatment

| Treatment Group Comparison | Odds Ratio | P-value |
|---|---|---|
| IQNPA + IQNLF vs. IQNLF | 15.00 | 0.0002* |
| Log₁₀(IQNLF Dose) | 0.98 | 0.9788 |

Overall, the results of this study suggest that there is no significant dose-response relationship. However, there is a significant treatment effect with the combined antibody (IQNPA+IQNLF) treatment resulting in a higher probability of survival. The group that received no treatment (group 6) was significantly less protected than all treatment groups except groups treated with 10 mg/kg IQNLF (group 1), 7.5 mg/kg IQNLF (group 2), and 2.5 mg/kg IQNLF (group 4) when time to death was considered in addition to the overall survival rates. Groups 8, 10, and 11 which were treated with both antibodies and where most animals survived also showed significantly greater protection than groups 1, 2, 3, and 5 which were treated only with IQNLF and where at most two animals survived.

IQNLF as stand alone treatment is not very protective. The IQNPA+IQNLF combination, however, is very successful in protecting from death due to anthrax challenge 24 hrs before treatment. The combination treatment provided a 15 times higher chance of survival in this post-exposure aerosol challenge model. Although the increased protection by the IQNPA+IQNLF combination was significant, there was no dose dependency (all but one combination group demonstrated 87.5% survival). The results of this study suggest that the IQNPA+IQNLF combination represents an improvement over the IQNLF treatment alone.

1.6.1.2 Presence of Bacteria in Blood

Table 12 illustrates the proportion of animals that were bacteremic at any time point post-challenge along with the 95 percent confidence interval. All animals surviving to study day 14 were negative for bacteria on study day 14. However, only 32% (11/34) of these surviving animals were positive at any time point. All except two animals that died or were euthanized prior to study day 14, were positive. While only 2.7% (1/36) of the animals were bacteremia positive on study day 7, 32.8% (25/76) of the animals were bacteremia positive on study day 2 and fifty-three percent (42/80) of the animals were bacteremia positive just prior to treatment.

The proportion of animals that were bacteremic at any time point in groups 4, 8, 9 and 11 was significantly lower than the pooled control group by Fisher's exact test. When the more stringent Bonferroni Holm adjustment was used to control the overall level of significance at 0.05, only group 8 was significantly different from controls (Table 12). There was a statistically significant correlation between testing positive for bacteria in the blood at any time point and death. Approximately 81 percent of animals that were bacteremic at any time point died and 100 percent of animals that died were bacteremic at some point. Table 13 is a frequency table that summarizes the relationship between an animal being bacteremic at any time point and death that includes day of death measurements. According to Fisher's two sided exact test of independence, the hypothesis that death and whether an animal was bacteremic at any time point was independent was rejected (p value<0.0001).

TABLE 12
Proportion of Animals that were Bacteremic at Any Time
Point and 95 Percent Binomial Confidence Interval

| | | | | | One-sided Fisher's Exact | |
|---|---|---|---|---|---|---|
| | | | | Proportion | P-value, Comparison | |
| | Treatment | | No. | Bacteremic | to Group 6 | |
| | Test | Dose | Bacteremic/ | (95% Confidence | | Bonferroni- |
| Group | Material | (mg/kg) | Total | Interval) | Unadjusted | Holm Adjusted |
| 1 | IQNLF | 10.0 | 7/8 | 0.88 (0.47, 1.00) | 0.4000 | 1.0000 |
| 2 | IQNLF | 7.5 | 6/8 | 0.75 (0.35, 0.97) | 0.1474 | 0.7368 |
| 3 | IQNLF | 5.0 | 7/8 | 0.88 (0.47, 1.00) | 0.4000 | 1.0000 |
| 4 | IQNLF | 2.5 | 5/8 | 0.63 (0.24, 0.91) | 0.0491* | 0.2947 |
| 5 | IQNLF | 1.25 | 4/4 | 1.00 (0.40, 1.00) | 1.0000 | 1.0000 |
| 6 | Control | PBS 1 mL/kg | 12/12 | 1.00 (0.74, 1.00) | | |
| 8 | IQNPA + IQNLF | 2.5 + 0.625* | 2/8 | 0.25 (0.03, 0.65) | 0.0007* | 0.0065* |
| 9 | IQNPA + IQNLF | 2.5 + 1.25* | 4/8 | 0.50 (0.16, 0.84) | 0.0144* | 0.1156 |
| 10 | IQNPA + IQNLF | 2.5 + 2.5* | 6/8 | 0.75 (0.35, 0.97) | 0.1474 | 0.7368 |
| 11 | IQNPA + IQNLF | 2.5 + 5.0* | 4/8 | 0.50 (0.16, 0.84) | 0.0144* | 0.1156 |

TABLE 13
Frequency Table of Animals Bacteremic at Any Time
Point Versus Survival Status (Alive or Dead)

Survival Status

| | | Survived | Died |
|---|---|---|---|
| Bacteremia | Positive | 11 | 46 |
| | Negative | 23 | 0 |

1.6.1.3 Clinical Observations

Clinical observations were documented from day 0 through day 14 or time of death. Lethargy, stool abnormalities (soft stool, diarrhea, and no stool), and lack of eating were the most common clinical observations noted during the post-challenge observation period. All of the control animals displayed clinical symptoms including not eating, lethargy or no stool from day 2 post-challenge until death. All animals receiving 10.00, 7.50, 5.00, 2.50, or 1.25 mg/kg IQNLF displayed clinical symptoms such as not eating, lethargy, lacrimation, soft stool, labored breathing, and/or no stool as early as Study Day 2. While animals receiving the combination treatment displayed clinical symptoms such as not eating, lacrimation, soft stool, and lethargy, the symptoms themselves as well as the length of time the symptoms last, was shortened considerably. By study day 8, 14 out of the 16 remaining animals in these groups were completely normal.

1.6.2 Methods

Test System: Eighty (40 male and 40 female) specific pathogen free New Zealand white rabbits (purchased from Covance Laboratories), weighing between 2.72 to 3.96 kg at the time of randomization and which were in good health were placed on study. Eighty rabbits were ordered, therefore, there would be no replacements in the event that a rabbit was to be removed from the study.

Aerosol Challenge: This study required two aerosol challenge days with 40 rabbits challenged per day. The first 40 rabbits were challenged and treated and followed for 14 days and then the second 40 rabbits arrived, were challenged, treated, and followed for 14 days. Thus, there were two separate randomizations performed. Prior to challenge day A, rabbits were assigned to one of six groups based on animal study day −8 weights, and a challenge order per day. The day of aerosol challenge was considered Day 0. The first group of 40 rabbits was randomized according to Table 14A and the second group of 40 rabbits was randomized according to Table 14B. All rabbits to be challenged on the second of the two challenge days were randomized by Study Day −7 weights. Rabbits were transported into the BL3 facility immediately upon arrival for quarantine. On Study Day 0, rabbits were placed into a plethysmography chamber and passed into a Class III cabinet system, and challenged with a targeted aerosol dose of 100 LD50s B. anthracis (Ames strain) spores. The concentrations of B. anthracis inhaled by the rabbits was determined from the number of B. anthracis spores collected directly from an animal exposure port by an in-line impinger (Model 7541, Ace Glass Incorporated). Serial dilutions of impinger samples were plated and enumerated. The inhaled dose was calculated using the number of CFU/liter of air multiplied by the respiratory volume of the rabbits. The overall average dose for the two aerosol challenge days was 91±27 LD50s with an average challenge dose of 115±34 LD50s for the first day of challenges and an average dose of 66±19 LD50s for the second day of challenges. The mass-median aerodynamic diameter for challenge material aerosols on day one was 1.18 μm and the mass-median aerodynamic diameter for challenge material aerosols on day two was 1.15 μm (as determined with an Aerodynamic Particle Sizer (APS model 3321, TSI Inc, St. Paul, Minn.)).

TABLE 14A
Study Design Part I

| | | | | | Time of | |
|---|---|---|---|---|---|---|
| | | Rabbits | | Treatment | Treatment^{b} | Blood Draw |
| Group | | Per | Treatment | Dose | (Hours Post- | Schedule |
| ID | Treatment | Group | Route | (mg/kg)^{a} | Challenge) | (Study Day) |
| 1 | IQNLF | 8 | I.V. | 10 | +24 | −7, 1^{c}, 2, 7, 14 or TOD |
| 2 | IQNLF | 8 | I.V. | 7.5 | +24 | −7, 1^{c}, 2, 7, 14 or TOD |
| 3 | IQNLF | 8 | I.V. | 5 | +24 | −7, 1^{c}, 2, 7, 14 or TOD |
| 4 | IQNLF | 8 | I.V. | 2.5 | +24 | −7, 1^{c}, 2, 7, 14 or TOD |
| 5 | IQNLF | 4 | I.V. | 1.25 | +24 | −7, 1^{c}, 2, 7, 14 or TOD |
| 6 | PBS Only | 4 | I.V. | 1 mL/kg | +24 | −7, 1^{c}, 2, 7, 14 or TOD |

^{a}Single bolus dose
^{b}Treatment time to be 24 hours post-exposure ± 15 minutes
^{c}Blood to be collected immediately prior to treatment

TABLE 14B
Study Design Part II

| | | | | | Time of | |
|---|---|---|---|---|---|---|
| | | Rabbits | | Treatment | Treatment^{b} | Blood Draw |
| Group | | Per | Treatment | Dose | (Hours Post- | Schedule |
| ID | Treatment | Group | Route | (mg/kg)^{a} | Challenge) | (Study Day) |
| 7 | PBS Only* | 4 | I.V. | 1 mL/kg | +24 | −7, 1^{c}, 2, 7, 14 or TOD |
| 8 | IQNPA + IQNLF | 8 | I.V. | 2.5 + 0.625^{d} | +24 | −7, 1^{c}, 2, 7, 14 or TOD |
| 9 | IQNPA + IQNLF | 8 | I.V. | 2.5 + 1.25^{d} | +24 | −7, 1^{c}, 2, 7, 14 or TOD |
| 10 | IQNPA + IQNLF | 8 | I.V. | 2.5 + 2.5^{d} | +24 | −7, 1^{c}, 2, 7, 14 or TOD |
| 11 | IQNPA + IQNLF | 8 | I.V. | 2.5 + 5.0^{d} | +24 | −7, 1^{c}, 2, 7, 14 or TOD |
| 12 | PBS Only | 4 | I.V. | 1 mL/kg | +24 | −7, 1^{c}, 2, 7, 14 or TOD |

^{a}Single bolus dose
^{b}Treatment time to be 24 hours post-exposure ± 15 minutes
^{c}Blood to be collected immediately prior to treatment
^{d}IQNPA dose concentration remained the same at 2.5 mg/kg while the IQNLF dose varied accordingly
*See Deviation (DR-5813)

Post-Exposure Dosing: Approximately twenty-four hours post-challenge with B. anthracis, the rabbits were administered antibodies according to Tables 14A and B. Doses were given via a single bolus intravenous injection.

Blood Collection: Blood samples were collected according to Tables 14A and B. Blood was drawn from the marginal ear vein according to SOP BBRC.VII-020. Oil of wintergreen (topical) or acepromazine (1-5 mg/kg subcutaneously) was utilized to facilitate blood sampling via the ear. Amounts of blood collected fell within the guidelines established by the Battelle IACUC, derived in part from the Canadian Guide to the Care and Use of Experimental Animals.

Bacteremia (Culture): Blood collected in EDTA tubes on days −7, just prior to treatment, 2, 14 and/or at time of death was cultured by streaking ˜40 μl of whole blood over blood ager plates, to determine the presence or absence of B. anthracis bacteremia.

Sera Collection and Shipment: Approximately 2.0 ml of whole blood was collected into SST tubes on study days −7, 14 or at time of death. This blood was processed and the serum collected. Serum was then filtered and checked for sterility for shipment to IQ Corporation for serological analysis. When possible, a terminal sample was taken from any animal found dead or found to be moribund prior to euthanasia.

Clinical Observations: Animals were monitored twice daily by laboratory animal personnel near the beginning and end of each workday for abnormal clinical signs (such as respiratory distress, inappetence, inactivity, seizures and moribundity) until Study Day 14. Any rabbits that were moribund, as assessed by a highly trained life sciences technician, Battelle veterinarian, or Study Director, were euthanized.

### 1.7 Post-Exposure Efficacy

### Experiment 3

The objective of this study was to determine whether treatment with the IQNPA and IQNLF antibodies, alone or in combination, could extend the window for treatment following inhalational infection with B. anthracis. The IQNPA and IQNLF antibodies were given alone or in combination at 24, 32, 40, and 48 hours post-challenge. The target infectious dose for this study was 100 LD50s; the overall average dose for the three study days was 100±25 LD50s with an average challenge dose of 91±28 LD50s for the first day of challenges and an average dose of 102±19 LD50s for the second day of challenges and an average dose of 106±29 LD50s for the third day of challenge.

While survival was the key objective of this study, several other parameters including temperature, body weight, clinical observations, and bacteremia were also examined.

1.7.1 Results

1.7.1.1 Survival

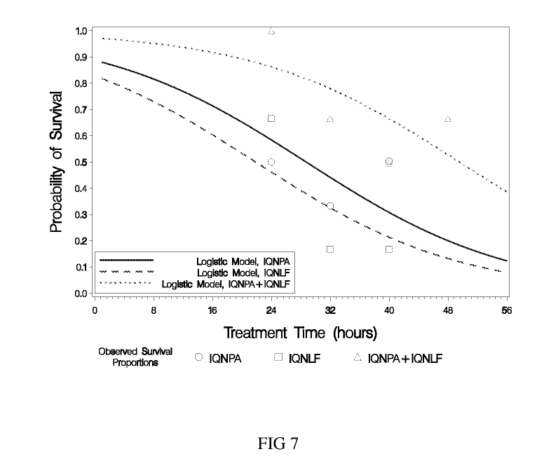

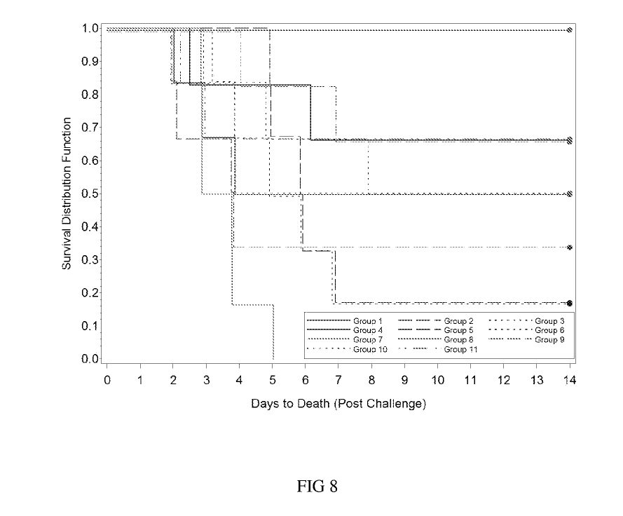

The results from the logistic regression model fitted to the survival data shows that there was a significant treatment effect. Groups treated with the combination of IQNPA and IQNLF antibodies had significantly greater odds of survival than either antibody alone (FIG. 7). Fisher's exact test showed that three of the groups treated with both IQNPA and IQNLF antibodies (groups 8, 9, and 11) had significantly greater survival rates than the control group. When a Bonferroni Holm adjustment was used to control the overall level of significance at 0.05, only the group treated with IQNPA+IQNLF at treatment time 24 hrs (group 8) was significantly different from the control group (group 7). FIG. 8 shows the Kaplan-Meier survival curves for each group.

TABLE 15
Dosing Schedule

| | IQNPA | IQNLF | IQNPA + IQNLF |
|---|---|---|---|
| Group nr | 2.5 mg/kg @ | 7.5 mg/kg @ | 2.5 + 7.5 mg/kg @ |
| 1 | +24 hrs | — | — |
| 2 | +32 hrs | — | — |
| 3 | +40 hrs | — | — |
| 4 | — | +24 hrs | — |
| 5 | — | +32 hrs | — |
| 6 | — | +40 hrs | — |
| 7 | PBS control (1 mL/kg) @ +24 hrs | | |
| 8 | — | — | +24 hrs |
| 9 | — | — | +32 hrs |
| 10 | — | — | +40 hrs |
| 11 | — | — | +48 hrs |

The log rank test applied to the time to death data showed a statistically significant difference over the control group for Group 5 (IQNLF at 32 hrs), Group 8 (IQNPA+IQNLF at 24 hrs), and Group 9 (IQNPA+IQNLF at 32 hrs) when time to death was considered in addition to the overall survival rates and a Bonferroni Holm adjustment was used to control the overall level of significance at 0.05.

TABLE 16
Survival Rate and Results of Fisher's Exact Test Comparison for Each Treatment Group

| | | | | | | One-sided Fisher's Exact | |
|---|---|---|---|---|---|---|---|
| | Treatment | | | | | P-value, Comparison | |
| | | | Treatment | No. | Survival Rate | to Group 7 | |
| | Test | Dose | Time | Survived/ | (95% Confidence | | Bonferroni- |
| Group | Material | (mg/kg) | (hours) | Total | Interval) | Unadjusted | Holm Adjusted |
| 1 | IQNPA | 2.5 | 24 | 3/6 | 0.50 (0.12, 0.88) | 0.0909 | 0.5455 |
| 2 | IQNPA | 2.5 | 32 | 2/6 | 0.33 (0.04, 0.78) | 0.2273 | 0.6818 |
| 3 | IQNPA | 2.5 | 40 | 3/6 | 0.50 (0.12, 0.88) | 0.0909 | 0.5455 |
| 4 | IQNLF | 7.5 | 24 | 4/6 | 0.67 (0.22, 0.96) | 0.0303* | 0.2727 |
| 5 | IQNLF | 7.5 | 32 | 1/6 | 0.17 (0.00, 0.64) | 0.5000 | 1.0000 |
| 6 | IQNLF | 7.5 | 40 | 1/6 | 0.17 (0.00, 0.64) | 0.5000 | 1.0000 |
| 7 | Control | PBS Alone | 24 | 0/6 | 0.00 (0.00, 0.46) | | |
| 8 | IQNPA + IQNLF | 2.5 + 7.5 | 24 | 6/6 | 1.00 (0.54, 1.00) | 0.0011* | 0.0108* |
| 9 | IQNPA + IQNLF | 2.5 + 7.5 | 32 | 4/6 | 0.67 (0.22, 0.96) | 0.0303* | 0.2727 |
| 10 | IQNPA + IQNLF | 2.5 + 7.5 | 40 | 3/6 | 0.50 (0.12, 0.88) | 0.0909 | 0.5455 |
| 11 | IQNPA + IQNLF | 2.5 + 7.5 | 48 | 4/6 | 0.67 (0.22, 0.96) | 0.0303* | 0.2727 |

TABLE 17
Percent Survival

| | | Treatment | | |
|---|---|---|---|---|
| | | Time (Hrs post- | | Percent |
| Group | Treatment | challenge) | Dose (mg/kg) | Survival |
| 1 | IQNPA | 24 | 2.5 | 50 |
| 2 | IQNPA | 32 | 2.50 | 33 |
| 3 | IQNPA | 40 | 2.5 | 50 |
| 4 | IQNLF | 24 | 7.5 | 66 |
| 5 | IQNLF | 32 | 7.50 | 16 |
| 6 | IQNLF | 40 | 7.5 | 16 |
| 7 | Control | 24 | 1 mL/kg | 0 |
| 8 | IQNPA + IQNLF | 24 | 2.5 + 7.5 | 100 |
| 9 | IQNPA + IQNLF | 32 | 2.5 + 7.5 | 66 |
| 10 | IQNPA + IQNLF | 40 | 2.5 + 7.5 | 50 |
| 11 | IQNPA + IQNLF | 48 | 2.5 + 7.5 | 66 |

TABLE 18
Unadjusted p-values from the pairwise log-rank test comparing
time-to-death and overall survival between all groups

| Group | 1 | 2 | 3 | 4 | 5 | 6 | 7 | 8 | 9 | 10 |
|---|---|---|---|---|---|---|---|---|---|---|
| 2 | 0.5213 | | | | | | | | | |
| 3 | 0.7426 | 0.3468 | | | | | | | | |
| 4 | 0.5126 | 0.1967 | 0.6475 | | | | | | | |
| 5 | 0.6703 | 0.7054 | 0.3271 | 0.1171 | | | | | | |
| 6 | 0.5759 | 0.8336 | 0.2578 | 0.1171 | 0.6451 | | | | | |
| 7 | 0.1093 | 0.3685 | 0.0117* | 0.0111* | 0.0046* | 0.0271* | | | | |
| 8 | 0.0554 | 0.0183* | 0.0554 | 0.1385 | 0.0043* | 0.0043* | 0.0005* | | | |
| 9 | 0.3912 | 0.1265 | 0.5901 | 0.9193 | 0.0817 | 0.0576 | 0.0016* | 0.1385 | | |
| 10 | 0.9467 | 0.4297 | 0.8065 | 0.5126 | 0.6703 | 0.5097 | 0.0486* | 0.0554 | 0.3912 | |
| 11 | 0.5901 | 0.2955 | 0.7206 | 0.9193 | 0.2387 | 0.2387 | 0.0498* | 0.1385 | 0.8395 | 0.6606 |

1.7.1.2 Presence of Bacteria in Serum

58% (38/66) of the rabbits in this study were bacteremic at some point (either during the study, or at the time of death) while the remaining 42% (28/66) was never blood culture positive at any time during the study. All rabbits were exposed to a 100 LD50 aerosol dose (based on the plate counts for the impinger samples taken during the exposure process). Thus, it is unlikely that they were not infected. The negative blood culture results are most likely due to a concentration of bacteria at the time of collection that was below the limits of detection (CFU/ml). Unless the IQNPA and/or IQNLF antibodies can completely eliminate both bacteria and toxin, one would expect to see a bacteremia positive result at some point during the study; typically early after infection. However, these results may indicate that the antibodies were able to suppress bacterial growth in the blood below the level of detection. The inability to detect bacteria in the blood precluded a determination of whether the antibodies, alone or the combination, were able to clear the bacteria.

Table 19 summarizes the bacteremia results for all animals of this study. Twenty-nine percent (21/72) of the challenged animals were bacteremic on day 1. All animals that survived to study day 14 were negative. 16% (5/31) of these surviving animals had positive blood cultures at any time during the study. In contrast, all except three of the animals which died or were euthanized prior to study day 14 were bacteremic. Three of the six animals (50%) that received 2.5 mg/kg IQNPA 24 hours post-challenge were blood culture negative on study day 14 while only 2/6 and 3/6 of the animals receiving 2.5 mg/kg IQNPA 32 and 40 hours post-exposure were blood culture negative at the end of the study. Sixty-six percent (4/6) of the rabbits receiving 7.5 mg/kg IQNLF 24 hours post-challenge were blood culture negative at the end of the study (day 14). Only one rabbit from each of the other two IQNLF treatment groups survived and both were blood culture negative. One hundred percent (6/6) of the rabbits receiving the combination treatment 24 hours post-challenge were blood culture negative at the end of the study. Sixty-six percent (4/6), 50% (3/6), and 66% (4/6) of the rabbits receiving the combination treatment 32, 40, and 48 hours post-challenge were blood culture negative at the end of the study.

TABLE 19
Proportion of Animals that were Bacteremic at Any Time Point and 95% Binomial Confidence Interval

| | | | | | | One-sided Fisher's Exact | |
|---|---|---|---|---|---|---|---|
| | Treatment | | | | Proportion | P-value, Comparison | |
| | | | Treatment | No. | Bacteremic | to Group 7 | |
| | Test | Dose | Time | Bacteremic/ | (95% Confidence | | Bonferroni- |
| Group | Material | (mg/kg) | (hours) | Total | Interval) | Unadjusted | Holm Adjusted |
| 1 | IQNPA | 2.5 | 24 | 3/6 | 0.50 (0.12, 0.88) | 0.2727 | 1.0000 |
| 2 | IQNPA | 2.5 | 32 | 4/6 | 0.67 (0.22, 0.96) | 0.5000 | 1.0000 |
| 3 | IQNPA | 2.5 | 40 | 3/6 | 0.50 (0.12, 0.88) | 0.2727 | 1.0000 |
| 4 | IQNLF | 7.5 | 24 | 3/6 | 0.50 (0.12, 0.88) | 0.2727 | 1.0000 |
| 5 | IQNLF | 7.5 | 32 | 5/6 | 0.83 (0.36, 1.00) | 0.7727 | 1.0000 |
| 6 | IQNLF | 7.5 | 40 | 5/6 | 0.83 (0.36, 1.00) | 0.7727 | 1.0000 |
| 7 | Control | PBS Alone | 24 | 5/6 | 0.83 (0.36, 1.00) | | |
| 8 | IQNPA + IQNLF | 2.5 + 7.5 | 24 | 3/6 | 0.50 (0.12, 0.88) | 0.2727 | 1.0000 |
| 9 | IQNPA + IQNLF | 2.5 + 7.5 | 32 | 2/6 | 0.33 (0.04, 0.78) | 0.1212 | 1.0000 |
| 10 | IQNPA + IQNLF | 2.5 + 7.5 | 40 | 3/6 | 0.50 (0.12, 0.88) | 0.2727 | 1.0000 |
| 11 | IQNPA + IQNLF | 2.5 + 7.5 | 48 | 2/6 | 0.33 (0.04, 0.78) | 0.1212 | 1.0000 |

1.7.1.3 Clinical Observations

Clinical observations were recorded from day 0 through day 14 or time of death. Lethargy, stool abnormalities (soft stool, diarrhea, and no stool), and lack of eating were the most common clinical observations noted during the post-challenge observation period. One hundred percent of the control animals displayed clinical symptoms including not eating, lethargy, no stool, and lacrimation from day 3 post-challenge until death. For Groups 1 (2.5 mg/kg IQNPA at 24 hrs) and 2 (2.5 mg/kg IQNPA at 32 hrs), 66% (4/6) of animals displayed clinical symptoms on 3/14 days during the post-challenge observation period. For Group 3 (2.5 mg/kg IQNPA at 40 hrs), 100% (6/6) of animals displayed clinical symptoms on 6/14 days. For Group 8 (IQNPA+IQNLF at 24 hrs), 50% (3/6) of animals displayed clinical symptoms on 9/14 days. For Group 9 (IQNPA+IQNLF at 32 hrs), 66% (4/6) of animals displayed clinical symptoms on 6/14 days. For Group 10 (IQNPA+IQNLF at 40 hrs), 83% (5/6) of animals displayed clinical symptoms on 12/14 days. For Group 11 (IQNPA+IQNLF at 48 hrs), 66% (4/6) of animals displayed clinical signs on 10/14 days.

1.7.2 Methods

Test System: Seventy-two (36 male and 36 female) New Zealand white rabbits (purchased from Covance Laboratories), specific pathogen free (SPF), that weighed between 2.0 to 4.0 kg at the time of randomization and were in good health were placed on study. Seventy-eight rabbits were ordered. As all animals were free of malformations and illness, the replacement animals were not required, and were transferred to a training protocol and used for training purposes.

Aerosol Challenge: This study required three aerosol challenge days with 22 rabbits challenged per day. Rabbits were transported into the BL-3 facility 3 days prior to challenge to allow time for acclimation. On Study Day 0, rabbits were placed into a plethysmography chamber and passed into a Class III cabinet system, and challenged with a targeted aerosol dose of 100 LD50s B. anthracis (Ames strain) spores. The concentrations of B. anthracis inhaled by the rabbits is determine from the number of B. anthracis collected directly from an animal exposure port by an in-line impinger (Model 7541, Ace Glass Incorporated). Serial dilutions of impinger samples were plated and enumerated as per SOP BBRC.X-054. The inhaled dose was calculated using the number of CFU/liter of air multiplied by the respiratory volume of the rabbits. The overall average dose for the three study days was 100±25 LD50s with an average challenge dose of 91±28 LD50s for the first day of challenges and an average dose of 102±19 LD50s for the second day of challenges and an average dose of 106±29 for the third challenge day. The mass-median aerodynamic diameter for challenge material aerosols on day one was 1.16 μm and the mass-median aerodynamic diameter for challenge material aerosols on day two was 1.15 μm and the mass-median aerodynamic diameter for challenge material aerosols on day three was 1.12 μm (as determined with an Aerodynamic Particle Sizer (APS model 3321, TSI Inc, St. Paul, Minn.)).

Post-Exposure Dosing: Approximately twenty-four hours post-challenge with B. anthracis, the rabbits were administered either IQNPA (2.5 mg/kg), IQNLF (7.5 mg/kg), or a combination of the two (see Table 15, supra). Doses were given via a single bolus intravenous injection. Briefly, groups one through three received 2.5 mg/kg of IQNPA at the indicated times post-challenge, group six received buffer only as a control, and groups eight through eleven received 7.5 mg/kg IQNLF at 24, 32, 40, and 48 hours post-challenge. All rabbits were treated according to Table 15. The study director visually verified doses prior to being administered to ensure the required dose levels were given. Rabbits were administered 2.5 mg/kg IQNPA at 24, 32, and 40 hours post-challenge; 7.5 mg/kg IQNLF was administered at 24, 32, and 40 post-challenge; 5.0+7.5 mg/kg IQNPA+IQNLF was administered 24, 32, 40, and 48 hours after being challenged with B. anthracis. All treatments were via a single bolus dose.

Temperature Monitoring: Body temperatures were monitored twice daily via an implantable programmable temperature transponder chip (IPTT-300, BMDS, Seaford, Del.). Temperature chips were implanted on or before study day −9 depending on which day the animals were to be challenged. Rabbits were sedated with acepromazine (1-5 mg/kg) prior to implantation of the transponder chips and each rabbit had two chips injected subcutaneously (one at shoulder blade level and one at rump level). Recording of twice daily baseline body temperature from both transponder chips began on or before study day −11 and continued until the morning of each groups day of challenge. Clinical temperature readings began in the afternoon of the day of challenge and were taken twice daily for the duration of the study. Post-challenge body temperatures were monitored from a single transponder chip (rump). All temperature readings were taken prior to treatment with acepromazine.

Animal Weights: Animals were weighed once daily for the duration of the study beginning 10 days prior to the first challenge day. Weights were used to determine the amount of acepromazine to be administered prior to temperature transponder implantation. Study day 0 weights were used to determine the required amount of test/control article to be administered.

Blood Collection: Blood samples were collected on time points −1, 0, 2, and 14 or time of death. Blood was drawn from the marginal ear vein. Oil of wintergreen (topical) or acepromazine (1-5 mg/kg subcutaneously) was utilized to facilitate blood sampling via the ear. Amounts of blood collected fell within the guidelines established by the Battelle IACUC, derived in part from the Canadian Guide to the Care and Use of Experimental Animals.

Bacteremia (Culture): Blood collected in EDTA tubes on days −1, just prior to treatment, 2, 14 and/or time of death were cultured, by streaking ˜40 μl of while blood over blood ager plates, to determine the presence or absence of B. anthracis bacteremia.

Sera Collection and Shipment: Approximately 2.0 ml of whole blood was collected into SST tubes (see Blood collection). This blood was processed and the serum collected. Serum was then filtered, and checked for sterility for shipment to IQ Corporation for serological analysis. When possible, a terminal sample was taken from any animal found dead or found to be moribund prior to euthanasia.

Clinical Observations: Animals were monitored twice daily by laboratory animal personnel near the beginning and end of each workday for abnormal clinical signs (such as respiratory distress, inappetence, inactivity, seizures and moribundity) until Study Day 14. Any rabbits that were moribund, as assessed by a highly trained life sciences technician, Battelle veterinarian, or Study Director, were euthanized.

### 1.8 Overall Survival Odds

An additional statistical analysis was performed to compare the data generated across the three different post-exposure studies, Post-Exposure Efficacy Experiments 1-3. The data used in this analysis included all groups treated with IQNPA, IQNLF, or combined IQNPA+IQNLF where the treatment was administered 24 hours post-challenge. The groups included in the analysis from each experiment are set forth in Tables 20-22 below.

TABLE 20
Groups Included from Post-Exposure Efficacy Experiment 1

| Group* | Treatment |
|---|---|
| 1 | IQNPA (5.0 mg/kg) |
| 2 | IQNPA (2.5 mg/kg) |
| 3 | IQNPA (1.25 mg/kg) |
| 4 | IQNLF (15 mg/kg) |
| 5 | IQNLF (7.5 mg/kg) |
| 6 | IQNLF (3.75 mg/kg) |
| 7 | Controls (PBS Alone) |
| 8 | IQNPA (5.0 mg/kg) + IQNLF (15 mg/kg) |
| 9 | IQNPA (2.5 mg/kg) + IQNLF (7.5 mg/kg) |
| 10 | IQNPA (1.25 mg/kg) + IQNLF (3.75 mg/kg) |
| 11 | IQNPA (0.625 mg/kg) + IQNLF (1.88 mg/kg) |
| 12 | IQNPA (0.3125 mg/kg) + IQNLF (0.94 mg/kg) |

TABLE 21
Groups Included from Post-Exposure Efficacy Experiment 2

| Group* | Antibody | Dose | | Number of Animals |
|---|---|---|---|---|
| 1 | IQNLF | 10.0 | mg/kg IQNLF | 8 |
| 2 | IQNLF | 7.5 | mg/kg IQNLF | 8 |
| 3 | IQNLF | 5.0 | mg/kg IQNLF | 8 |
| 4 | IQNLF | 2.5 | mg/kg IQNLF | 8 |
| 5 | IQNLF | 1.25 | mg/kg IQNLF | 4 |
| 6 | PBS (Control) | 1 | mL/kg | 4 |
| 8 | IQNPA + IQNLF | 2.5 mg/kg IQNPA + 0.625 mg/kg IQNLF | | 8 |
| 9 | IQNPA + IQNLF | 2.5 mg/kg IQNPA + 1.25 mg/kg IQNLF | | 8 |
| 10 | IQNPA + IQNLF | 2.5 mg/kg IQNPA + 2.5 mg/kg IQNLF | | 8 |
| 11 | IQNPA + IQNLF | 2.5 mg/kg IQNPA + 5.0 mg/kg IQNLF | | 8 |
| 12 | PBS (Control) | 1 | mL/kg | 8 |

TABLE 22
Groups Included from Post-Exposure Efficacy Experiment 3
(highlighted in grey)

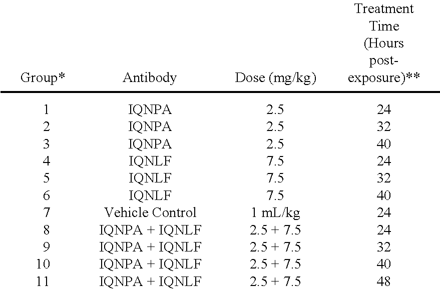

Two logistic regression models were fitted to the survival data for all groups with 24 hour post-challenge vaccinations across the three studies (no controls). The first modeled survival against the base-10 log-transformed IQNLF dose, an indicator for treatment where treatment is defined as either the IQNLF antibody or the combined IQNPA and IQNLF antibodies. The second modeled survival against the base-10 log-transformed IQNPA dose, an indicator for treatment where treatment is defined as either the IQNPA antibody or the combined IQNPA and IQNLF antibodies.

1.8.1 Results

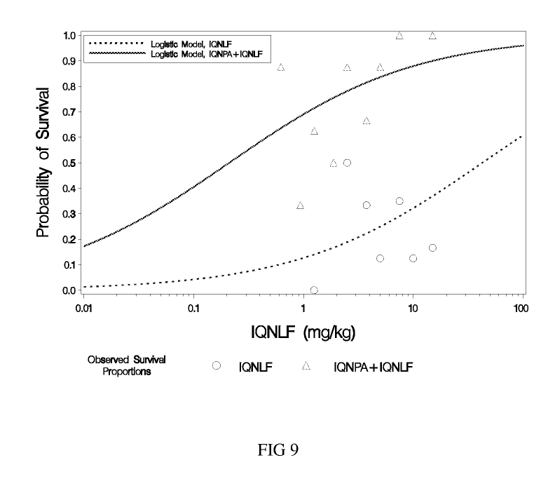

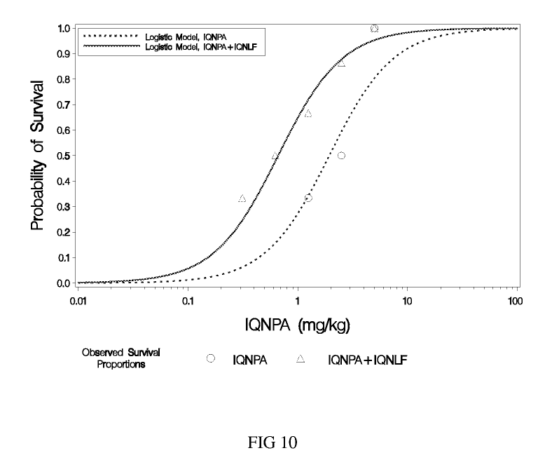

The results indicate that there were significant differences between treatment groups and controls in Experiments 1 and 2 and that there was a dose-response in Experiment 1, but not in experiment 2. Thus, the result show that there was a significant dose effect in the study where both IQNPA and IQNLF dosages were variable in the combination (Experiment 1), but not in the study where the IQNPA dose was fixed and the IQNLF dose was varied (Experiment 2). Importantly, the results also indicate that the odds of survival for animals treated with both antibodies (IQNPA+IQNLF) are significantly higher than for animals treated with either antibody alone. When compared to the IQNPA antibody, the odds of survival are 5 times higher and when compared with the IQNLF antibody, the odds of survival are 15 times higher. FIGS. 9 and 10 show the estimated logistic regression curves for each treatment.

TABLE 23
Effects included in Logistic Regression
Model Fitted to IQNLF Dose

| Effect | P-value |
|---|---|
| Intercept | 0.0008* |
| Treatment | <0.0001* |
| Log₁₀(IQNLF Dose) | 0.0562 |

*Effect significant at the 0.05 level of significance

TABLE 24
Summary of Odds Ratios from Logistic
Regression Model Fitted to IQNLF Dose

| Treatment Group | | |
|---|---|---|
| Comparison | Odds Ratio | P-value |
| (IQNPA + IQNLF) vs IQNLF | 15.327* | <0.0001 |
| Log₁₀(IQNLF Dose) | 3.268 | 0.0562 |

*Odds ratio significantly different from 1 at the 0.05 level of significance

TABLE 25
Effects included in Logistic Regression
Model Fitted to IQNPA Dose

| Effect | P-value |
|---|---|
| Intercept | 0.0751 |
| Treatment | 0.0068* |
| Log₁₀(IQNPA Dose) | 0.0001* |

*Effect significant at the 0.05 level of significance

TABLE 26
Summary of Odds Ratios from Logistic
Regression Model Fitted to IQNPA Dose

| Treatment Group | | |
|---|---|---|
| Comparison | Odds Ratio | P-value |
| (IQNPA + IQNLF) vs IQNPA | 4.918* | 0.0068 |
| Log₁₀(IQNPA Dose) | 30.635* | <0.0001 |

*Odds ratio significantly different from 1 at the 0.05 level of significance

Thus, the additional statistical analysis indicates that the combined IQNPA+IQNLF antibody treatment represents a statistically significant improvement over both treatment with either antibody alone.

### 1.9 Optimization of Dose-Response

The following describes a series of experiments to determine the optimal dosages for combination therapy. Initially, dose-response experiments will be performed to determine the smallest amount of anti-PA humAb IQNPA that gives the highest degree of protection against disease progression. Another set of dose-response experiments will then be performed using this optimal IQNPA dose along with varying doses of anti-LF (IQNLF) antibody to determine the optimal dose of IQNLF in combination with IQNPA. Finally, a third set of experiments will be performed to determine the optimal doses for the combination of IQNPA and IQNLF and Levofloxacin and to determine whether there are any negative side effects when the antibodies are combined with Levofloxacin. It is expected that no adverse effects will be manifest.

All experiments will be performed in 2-3 kilogram New Zealand White rabbits (F&M). The rabbits will be challenged with 200 times the LD50 equivalent of Bacillus anthracis Ames spores (200×LD50) by inhalation or intranasal instillation. Antibodies will be administered via a single injection of either (i) IQNPA alone, (ii) IQNLF alone, or (iii) either a single injection containing both IQNPA+IQNLF or two separate injections of IQNPA and IQNLF alone given at about the same time. Injections will be either subcutaneous, intramuscular, or intravenous. In addition, as a control, Levofloxacin (7 mg/kg/d for 6 consecutive days) will be administered at the same time as exposure to anthrax to demonstrate that the model can be modulated with agents other than monoclonal antibodies.

### 1.9.1 Example 1

### Optimization of IQNPA and Combination Dosages

Time of antibody treatment is the moment where the rabbits start to become symptomatic. The symptomatic phase (as measure by the presence of PA in blood and the presence of bacteremia) generally starts between 25 and 29 hrs after exposure. The results of these studies should demonstrate the added benefit of IQNLF with respect to survival.

Experiment 1: Dose finding for IQNPA (n = 8)

| | IQNPA i.v. | IQNLF i.v. | Time of |
|---|---|---|---|
| Group | (mg/kg) | (mg/kg) | treatment |
| 1 | saline | | Symptomatic |
| 2 | Levofloxacin 7 mg/kg/d i.m. | | Symptomatic |
| 3 | 2.5 | 0 | Symptomatic |
| 4 | 5 | 0 | Symptomatic |
| 5 | 7.5 | 0 | Symptomatic |
| 6 | 10 | 0 | Symptomatic |
| 7 | 12.5 | 0 | Symptomatic |
| 8 | 15 | 0 | Symptomatic |

Experiment 2: Use of optimal IQNPA dose
(A mg/kg) with different IQNLF dosages

| | | | |
|---|---|---|---|
| | IQNPA i.v. | IQNLF i.v. | Time of |
| Group | (mg/kg) | (mg/kg) | treatment |

| 1 | saline | | Symptomatic |
|---|---|---|---|
| 2 | Levofloxacin 7 mg/kg/d i.m. | | d 0 |
| 3 | A | 0 | Symptomatic |
| 4 | A | 0.5 | Symptomatic |
| 5 | A | 1.0 | Symptomatic |
| 6 | A | 2.0 | Symptomatic |
| 7 | A | 3.0 | Symptomatic |
| 8 | A | 4.0 | Symptomatic |
| 9 | A | 5.0 | Symptomatic |
| 10 | A | 7.5 | Symptomatic |
| 11 | A | 10.0 | Symptomatic |

Experiment 3: Optimal doses of IQNPA + IQNLF
(A mg/kg + B mg/kg) with Levofloxacin

| | IQNPA | IQNLF | | Levofloxacin | |
|---|---|---|---|---|---|
| | i.v. | i.v. | Time of | i.m. | Start of |
| Group | (mg/kg) | (mg/kg) | treatment | 7 mg/kg/d | Levofloxacin |
| 1 | saline | | Symptomatic | — | — |
| 2 | saline | | Symptomatic | for 6 days | d 0 |
| 3 | A | B | Symptomatic | — | — |
| 4 | A | B | Symptomatic | for 6 days | d 0 |

### 1.9.2 Example 2

### Combination with Levofloxacin

In this example, the start of the Levofloxacin treatment is at the time the rabbits become symptomatic. Time of antibody treatment is the moment where the rabbits start to become symptomatic. The symptomatic phase (as measure by the presence of PA in blood and the presence of bacteremia) generally starts between 25 and 29 hrs after exposure. The results should indicate that no significant negative impact of the antibiotic on the effect to the antibodies.

Experiment 1: Dose finding IQNPA + Levofloxacin (n = 8)

| | IQNPA i.v. | IQNLF i.v. | Time of |
|---|---|---|---|
| Group | (mg/kg) | (mg/kg) | treatment |
| 1 | saline | | Symptomatic |
| 2 | Levofloxacin 7 mg/kg/d i.m. | | Symptomatic |
| 3 | 2.5 | 0 | Symptomatic |
| 4 | 5 | 0 | Symptomatic |
| 5 | 7.5 | 0 | Symptomatic |
| 6 | 10 | 0 | Symptomatic |
| 7 | 12.5 | 0 | Symptomatic |
| 8 | 15 | 0 | Symptomatic |

Experiment 2: Use optimal IQNPA dose (A mg/kg)
and combine with different IQNLF dosages

| | | | |
|---|---|---|---|
| | IQNPA i.v. | IQNLF i.v. | Time of |
| Group | (mg/kg) | (mg/kg) | treatment |

| 1 | saline | | Symptomatic |
|---|---|---|---|
| 2 | Levofloxacin 7 mg/kg/d i.m. | | Symptomatic |
| 3 | A | 0 | Symptomatic |
| 4 | A | 0.5 | Symptomatic |
| 5 | A | 1.0 | Symptomatic |
| 6 | A | 2.0 | Symptomatic |
| 7 | A | 3.0 | Symptomatic |
| 8 | A | 4.0 | Symptomatic |
| 9 | A | 5.0 | Symptomatic |
| 10 | A | 7.5 | Symptomatic |
| 11 | A | 10.0 | Symptomatic |

Experiment 3: Use optimal IQNPA + IQNLF combination (A
mg/kg + B mg/kg) and combine with Levofloxacin

| | IQNPA | IQNLF | | Levofloxacin | |
|---|---|---|---|---|---|
| | i.v. | i.v. | Time of | i.m. | Start of |
| Group | (mg/kg) | (mg/kg) | treatment | 7 mg/kg/d | Levofloxacin |
| 1 | saline | | Symptomatic | — | — |
| 2 | saline | | Symptomatic | for 6 days | Symptomatic |
| 3 | A | B | Symptomatic | — | — |
| 4 | A | B | Symptomatic | for 6 days | Symptomatic |

### 1.9.3 Example 3

### Combination Antibodies and Levofloxacin Well into Symptomatic Phase (48 hrs)

Time of antibody treatment is the moment 48 hrs after exposure. The symptomatic phase (as measure by the presence of PA in blood and the presence of bacteremia) generally starts between 25 and 29 hrs after exposure, and 48 hrs is well into this symptomatic phase.

Experiment 1: Dose finding IQNPA + Levofloxacin(n = 8)

| | IQNPA i.v. | IQNLF i.v. | Time of |
|---|---|---|---|
| Group | (mg/kg) | (mg/kg) | treatment (hrs) |
| 1 | saline | | +48 |
| 2 | Levofloxacin 7 mg/kg/d i.m. | | +48 |
| 3 | 2.5 | 0 | +48 |
| 4 | 5 | 0 | +48 |
| 5 | 7.5 | 0 | +48 |
| 6 | 10 | 0 | +48 |
| 7 | 12.5 | 0 | +48 |
| 8 | 15 | 0 | +48 |

Experiment 2: Use optimal IQNPA dose (A mg/kg)
and combine with different IQNLF dosages

| | IQNPA i.v. | IQNLF i.v. | Time of |
|---|---|---|---|
| Group | (mg/kg) | (mg/kg) | treatment (hrs) |
| 1 | saline | | +48 |
| 2 | Levofloxacin 7 mg/kg/d i.m. | | +48 |
| 3 | A | 0 | +48 |
| 4 | A | 0.5 | +48 |
| 5 | A | 1.0 | +48 |
| 6 | A | 2.0 | +48 |
| 7 | A | 3.0 | +48 |
| 8 | A | 4.0 | +48 |
| 9 | A | 5.0 | +48 |
| 10 | A | 7.5 | +48 |
| 11 | A | 10.0 | +48 |

Experiment 3: Use optimal IQNPA + IQNLF combination (A
mg/kg + B mg/kg) and combine with Levofloxacin

| | IQNPA | IQNLF | | Levofloxacin | |
|---|---|---|---|---|---|
| | i.v. | i.v. | Time of | i.m. | Start of |
| Group | (mg/kg) | (mg/kg) | treatment | 7 mg/kg/d | Levofloxacin |
| 1 | saline | | +48 | — | — |
| 2 | saline | | +48 | for 6 days | +48 |
| 3 | A | B | +48 | — | — |
| 4 | A | B | +48 | for 6 days | +48 |

### 1.10 Post-Exposure Efficacy

### Experiment 4

The objective of this study was to determine whether treatment with the IQNPA alone or in combination with IQNLF could extend the window for treatment following nasal instillation with B. anthracis. The IQNPA was given alone or in combination with IQNLF to New Zealand rabbits at 48 hours post-challenge. The target infectious dose for this study was 100 LD50s. Survival of rabbits was examined.

1.10.1 Results

1.10.1.1 Survival

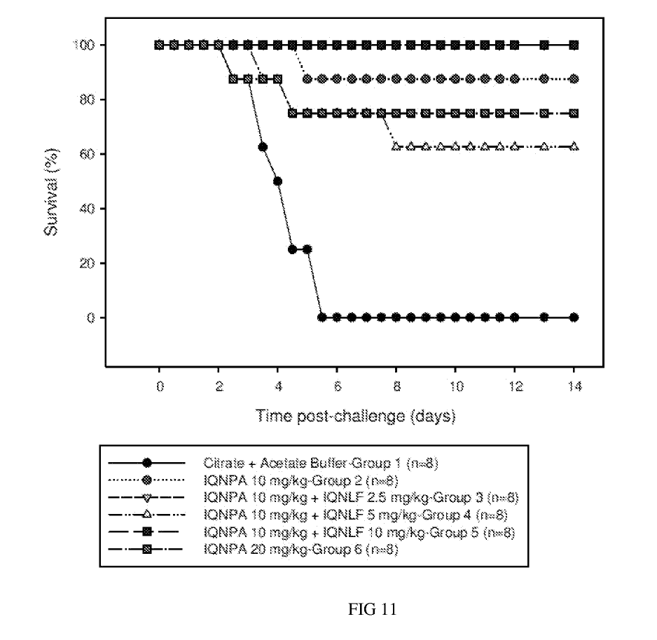

Survival rate was shown in Table 27 and FIG. 11.

TABLE 27
Percent Survival

| | | Treatment | Dose | |
|---|---|---|---|---|
| | | Time (Hrs post- | (IQNPA/IQNLF) | Percent |
| Group | Treatment | challenge) | (mg/kg) | Survival |
| 1 | Control | 48 | — | 0 |
| 2 | IQNPA | 48 | 10/— | 87.5 |
| 3 | IQNPA/IQNLF | 48 | 10/2.5 | 100 |
| 4 | IQNPA/IQNLF | 48 | 10/5 | 62.5 |
| 5 | IQNPA/IQNLF | 48 | 10/10 | 100 |
| 6 | IQNPA | 48 | 20/— | 75 |

1.10.2 Methods

Test System: New Zealand white rabbits (specific pathogen free (SPF)) purchased from Covance Laboratories were in good health, weighed and randomized, and were placed on study. Forty-eight rabbits were ordered. All animals were free of malformations and illness.

Nasal Instillation: B. anthracis spore challenge was carried out as described in Peterson J. W., et al, Infenction and Immunity 75, 3414-3424. Briefly, rabbits were transported into the BL-3 facility 3 days prior to challenge to allow time for acclimation. On Study Day 0, rabbits were placed into a plethysmography chamber and passed into a Class III cabinet system. Rabbits were anesthetized before nasal instillation and suspended vertically, using the upper incisors, with the bulk of the body weight of the rabbits resting on the base of a platform. The spore suspension was instilled slowly for 2 to 3 minutes onto the anterior opening of each naris. Subsequently, PBS was used to wash any nonadherent spores from the nasal cavity into the lungs of each rabbit.

Post-Exposure Dosing: Approximately forty-eight hours post-challenge with B. anthracis, the rabbits were administered IQNPA (10 or 20 mg/kg), either alone or with IQNLF at different doses (2.5, 5, or 10 mg/kg) (see Table 27, supra). Doses were given via a single bolus intravenous injection. Briefly, group one (control) received only the buffer; groups two and six received 10 mg/kg and 20 mg/kg of IQNPA, respectively; groups three to five received 10 mg/kg IQNPA+2.5 mg/kg IQNLF, 10 mg/kg IQNPA+5 mg/kg IQNLF and 10 mg/kg IQNPA+10 mg/kg IQNLF, respectively. The study director visually verified doses prior to administration to ensure the required dose levels were given.

### EQUIVALENTS

Those skilled in the art will recognize or be able to ascertain using no more than routine experimentation, many equivalents to the specific embodiments of the invention described herein. Such equivalents are intended to be encompassed by the following claims.

All references cited herein are incorporated herein by reference in their entirety and for all purposes to the same extent as if each individual publication or patent or patent application was specifically and individually indicated to be incorporated by reference in its entirety for all purposes.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

What is claimed is:

1. A method for the treatment of a disease caused by a bacterium, or B. anthracis toxins, toxin components, or homologs thereof, in a subject in need of such treatment comprising administering to the subject at least two monoclonal antibodies, or antigen binding fragments thereof, wherein each of the antibodies has affinity for a different epitope of a bacterial antigen selected from the protective antigen (PA), lethal factor (LF), and edema factor (EF) of B. anthracis, and a homolog thereof.

2. The method of claim 1, wherein the bacterium is selected from the group consisting of B. anthracis, B. cereus, B. thuringiensis, and C. perfringens.

3. The method of claim 1, wherein the disease is caused by toxemia from one or more bacterial toxins comprising one or more of PA, LF and EF, and a homolog thereof.

4. The method of claim 1, wherein the antibodies are human monoclonal antibodies.

5. The method of claim 1, wherein the antibodies are humanized monoclonal antibodies.

6. The method of claim 1, wherein the affinity (Kₐ) of the antibody for its antigen is from 10⁷ M⁻¹ to 10¹¹ M⁻¹.

7. The method of claim 6, wherein the affinity (Kₐ) of the antibody for its antigen is from 10⁹ M⁻¹ to 10¹⁰ M⁻¹.

8. The method of claim 1, wherein at least one antibody neutralizes the protective antigen of B. anthracis or a homolog thereof, or wherein at least one antibody neutralizes the lethal factor antigen of B. anthracis or a homolog thereof.

9. The method of claim 8, wherein the at least one antibody neutralizes the protective antigen of B. anthracis or a homolog thereof.

10. The method of claim 9, wherein the at least one antibody competitively inhibits the binding of a polypeptide comprising SEQ ID NO: 17 or 18 to the monoclonal antibody IQNPA.

11. The method of claim 9, wherein the at least one antibody comprises a variable heavy chain domain (VH) having three complementarity determining regions (CDR), each CDR comprising the following amino acid sequence: VH CDR1: KKPGA (SEQ ID NO: 5), VH CDR2: SNAIQWVRQAPGQRLEW (SEQ ID NO: 6), and VH CDR3: YMELSSLR (SEQ ID NO: 7).

12. The method of claim 9, wherein the at least one antibody comprises a variable light chain domain (VL) having three CDRs, each CDR comprising the following amino acid sequence: VL CDR1: LTQSPGTLSLS (SEQ ID NO: 8), VL CDR2: SYSSLAW (SEQ ID NO: 9), and VL CDR3: GPDFTLTIS (SEQ ID NO: 10).

13. The method of claim 9, wherein the at least one antibody comprises six CDRs, each comprising the following amino acid sequence: VH CDR1: SEQ ID NO: 5, VH CDR2: SEQ ID NO: 6, VH CDR3: SEQ ID NO: 7, VL CDR1: SEQ ID NO: 8, VL CDR2: SEQ ID NO: 9, and VL CDR3: SEQ ID NO: 10.

14. The method of claim 9, wherein the at least one antibody is the human monoclonal antibody IQNPA.

15. The method of claim 9, further comprising a second antibody which binds to the lethal factor antigen of B. anthracis or a homolog thereof.

16. The method of claim 15, wherein the second antibody competitively inhibits the binding of a protein comprising SEQ ID NO: 19 to the monoclonal antibody IQNLF.

17. The method of claim 15, wherein the second antibody comprises a VH having three CDRs, each CDR comprising the following amino acid sequence: VH CDR1: VQPGG (SEQ ID NO: 11), VH CDR2: SYAMSWVRQAPGKGLEW (SEQ ID NO: 12), and VH CDR3: YMQMNSL (SEQ ID NO: 13).

18. The method of claim 15, wherein the second antibody comprises a VL having three CDRs, each CDR comprising the following amino acid sequence: VL CDR1: TQSPDFQSVSP (SEQ ID NO: 14), VL CDR2: SSLHWYQ (SEQ ID NO: 15), and VL CDR3: DFTLTINSL (SEQ ID NO: 16).

19. The method of claim 15, wherein the second antibody comprises six CDRs, each comprising the following amino acid sequence: VH CDR1: SEQ ID NO: 11, VH CDR2: SEQ ID NO: 12, VH CDR3: SEQ ID NO: 13, VL CDR1: SEQ ID NO: 14, VL CDR2: SEQ ID NO: 15, and VL CDR3: SEQ ID NO: 16.

20. The method of claim 15, wherein the second antibody is the human monoclonal antibody IQNLF.

21. The method of claim 1, wherein each antibody is administered at a dose of from 1 to 20 mg/kg body weight of the subject.

22. The method of claim 21, wherein one antibody is administered at a dose of from 1 to 10 mg/kg body weight of the subject.

23. The method of claim 21, wherein one antibody is administered at a dose of from 2.5 to 15 mg/kg body weight of the subject.

24. The method of claim 1, wherein the antibodies are administered to the subject after the subject's exposure to the bacterium, or B. anthracis toxins, toxin components, or homologs thereof.

25. The method of claim 24, wherein the antibodies are administered to the subject before the subject develops any symptom after the exposure.

26. The method of claim 24, wherein the antibodies are administered to the subject after the subject develops a symptom.

27. The method of claim 1, further comprising administering to the subject an antibacterial agent.

28. The method of claim 27, wherein the antibacterial agent is levofloxacin.

29. A method for the prevention of a disease caused by a bacterium, or B. anthracis toxins, toxin components, or homologs thereof, in a subject in need of such prevention comprising administering to the subject at least two monoclonal antibodies, or antigen binding fragments thereof, wherein each of the antibodies has affinity for a different bacterial antigen selected from PA, LF, and EF, and a homolog thereof, and wherein the antibodies are administered at least 24 hours prior to the subject's exposure to the bacterium, or B. anthracis toxins, toxin components, or homologs thereof.

30. A pharmaceutical composition comprising at least two monoclonal antibodies, or antigen binding fragments thereof, wherein each of the antibodies has affinity for a different bacterial antigen selected from PA, LF, and EF, and a homolog thereof, and a pharmaceutically acceptable excipient or carrier.

31. The pharmaceutical composition of claim 30, wherein the composition comprises the monoclonal IQNPA antibody.

32. The pharmaceutical composition of claim 30, wherein the composition comprises the monoclonal IQNLF antibody.

33. The pharmaceutical composition of claim 30, wherein the composition comprises the monoclonal IQNPA antibody and the monoclonal IQNLF antibody.

34. The pharmaceutical composition of claim 30, further comprising at least one antibacterial agent.

35. The pharmaceutical composition of claim 34, wherein the at least one antibacterial agent is selected from ciprofloxacin, doxycycline, and levofloxacin.


SEQUENCE LISTING (19 sequences)

SEQ ID NO: 1 - protein, 468 aa, Homo sapiens
MDWIWRILFLVAAATGAHSQVQLVQSGAEVKKPGASVKVSCKASGYTFTSNAIQWVRQAPGQRLEWVGWINGGDGNTKYSQKFQGRVTISRDISASTAYMELSSLRSEDTAVYYCARHRLQRGGFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 235 aa, Homo sapiens
MEAPAQLLFLLLLWLPDTTGEIVLTQSPGTLSLSPGERATLSCRASQSVSYSSLAWYQQKPGQAPSLLIYGASSRATGIPDRFSGSGSGPDFTLTISRLEPEDFAVYYCQHYGNSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 470 aa, Homo sapiens
MELGLCWLFLVAILKGVQCEVQLLESGGGLVQPGGSLRLSCSGSGFMFSSYAMSWVRQAPGKGLEWVSGISGSGGTTNYADSVKGRFTISRDNSKNTLYMQMNSLRAEDTAVYYCAKDGVYGRLGGSDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 4 - protein, 233 aa, Homo sapiens
MLPSQLIGFLLLWVPASRGEIVLTQSPDFQSVSPKEKVTITCRASQSVGSSLHWYQQKPDQSPKLLIKYASQSFSGVPSRFSGSGSGTDFTLTINSLETEDAATYYCHQSSSLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 5 aa, Homo sapiens
KKPGA

SEQ ID NO: 6 - protein, 17 aa, Homo sapiens
SNAIQWVRQAPGQRLEW

SEQ ID NO: 7 - protein, 8 aa, Homo sapiens
YMELSSLR

SEQ ID NO: 8 - protein, 11 aa, Homo sapiens
LTQSPGTLSLS

SEQ ID NO: 9 - protein, 7 aa, Homo sapiens
SYSSLAW

SEQ ID NO: 10 - protein, 9 aa, Homo sapiens
GPDFTLTIS

SEQ ID NO: 11 - protein, 5 aa, Homo sapiens
VQPGG

SEQ ID NO: 12 - protein, 17 aa, Homo sapiens
SYAMSWVRQAPGKGLEW

SEQ ID NO: 13 - protein, 7 aa, Homo sapiens
YMQMNSL

SEQ ID NO: 14 - protein, 11 aa, Homo sapiens
TQSPDFQSVSP

SEQ ID NO: 15 - protein, 7 aa, Homo sapiens
SSLHWYQ

SEQ ID NO: 16 - protein, 9 aa, Homo sapiens
DFTLTINSL

SEQ ID NO: 17 - protein, 135 aa, Bacillus anthracis
NNIAVGADESVVKEAHREVINSSTEGLLLNIDKDIRKILSGYIVEIEDTEGLKEVINDRYDMLNISSLRQDGKTFIDFKKYNDKLPLYISNPNYKVNVYAVTKENTIINPSENGDTSTNGIKKILIFSKKGYEIG

SEQ ID NO: 18 - protein, 164 aa, Bacillus anthracis
TNIYTVLDKIKLNAKMNILIRDKRFHYDRNNIAVGADESVVKEAHREVINSSTEGLLLNIDKDIRKILSGYIVEIEDTEGLKEVINDRYDMLNISSLRQDGKTFIDFKKYNDKLPLYISNPNYKVNVYAVTKENTIINPSENGDTSTNGIKKILIFSKKGYEIG

SEQ ID NO: 19 - protein, 236 aa, Bacillus anthracis
ERNKTQEEHLKEIMKHIVKIEVKGEEAVKKEAAEKLLEKVPSDVLEMYKAIGGKIYIVDGDITKHISLEALSEDKKKIKDIYGKDALLHEHYVYAKEGYEPVLVIQSSEDYVENTEKALNVYYEIGKILSRDILSKINQPYQKFLDVLNTIKNASDSDGQDLLFTNQLKEHPTDFSVEFLEQNSNEVQEVFAKAFAYYIEPQHRDVLQLYAPEAFNYMDKFNEQEINLSLEELKDQ